(19) United States
(12) Patent Application Publication
(10) Pub. No.: US 2013/0149741 A1
(43) Pub. Date: Jun. 13, 2013

(54) METHODS FOR IMPROVED PRODUCTION OF BIOACTIVE WNT PROTEINS

(71) Applicants: Jennifer L. Green, Encinitas, CA (US); Geoffrey Wahl, San Diego, CA (US); Yao-Cheng Li, San Diego, CA (US)

(72) Inventors: Jennifer L. Green, Encinitas, CA (US); Geoffrey Wahl, San Diego, CA (US); Yao-Cheng Li, San Diego, CA (US)

(73) Assignee: SALK INSTITUTE FOR BIOLOGICAL STUDIES, La Jolla, CA (US)

(21) Appl. No.: 13/538,755

(22) Filed: Jun. 29, 2012

Related U.S. Application Data
(60) Provisional application No. 61/502,796, filed on Jun. 29, 2011.

(51) Int. Cl. C12P 21/00

(52) U.S. Cl. CPC C12P 21/00
USPC 435/69.1; 435/325

(57) ABSTRACT

Methods and compositions for protein expression are provided. In particular, cells producing efficient and reliable amounts of functional Wnt protein are provided.

### CROSS-REFERENCES TO RELATED APPLICATIONS

This application claims the benefit of U.S. Provisional Application No. 61/502,796 filed Jun. 29, 2011, which is hereby incorporated in its entirety and for all purposes.

### STATEMENT AS TO RIGHTS TO INVENTIONS MADE UNDER FEDERALLY SPONSORED RESEARCH AND DEVELOPMENT

This invention was made with government support under Federal Grant W81XWH-09-0326 awarded by USAMRMC. The Government has certain rights in the invention.

### REFERENCE TO A “SEQUENCE LISTING,” A TABLE, OR A COMPUTER PROGRAM LISTING APPENDIX SUBMITTED ON A COMPACT DISK

The Sequence Listing written in file 92150-006110US-842844_ST25.TXT, created on Jun. 29, 2012, 17,258 bytes, machine format IBM-PC, MS-Windows operating system, is hereby incorporated by reference in its entirety for all purposes.

### BACKGROUND OF THE INVENTION

Wnt signaling pathways are among the most important and most complex described in developmental biology. There are nineteen Wnt ligands that signal via receptors of the Frizzled family (ten members), Lrp co-receptors (two members) and receptor tyrosine kinases Ryk (one member) and Ror (two members) (reviewed by van Amerongen and Nusse 2009 [van Amerongen R, Nusse R, Development 136:3205-3214 (2009)]). In addition, there is an expanding repertoire of agonists and antagonists including R-spondins and their receptors Lgr4/5, which were recently found to associate with Frizzled and Lrp5/6 (Carmon K S et al., Proceedings of the National Academy of Sciences of the United States of America 108:11452-11457 (2011), de Lau W et al., Nature 476:293-297 (2011), Glinka A et al., EMBO reports 12:1055-1061 (2011)). In the canonical Wnt signaling pathway, Wnt binding to a Frizzled seven-pass transmembrane receptor and an Lrp5/6 co-receptor triggers a cascade of events resulting in accumulation and nuclear-translocation of the transcriptional activator beta-catenin. Alternative mechanisms of Wnt signaling, often referred to as “non-canonical,” do not stimulate beta-catenin-mediated gene transcription, but rather trigger changes in cell morphology, motility and polarity (Veeman M T et al., Developmental cell 5:367-377 (2003a)). These alternative mechanisms can involve the Ryk and/or Ror receptors and are often associated with repression of canonical Wnt signaling. While Wnts were historically divided into two classes, canonical and non-canonical, recent evidence suggests that canonical Wnts can be further sub-divided into three families based on their interaction with the Lrp co-receptor (Ettenberg S A et al., Proceedings of the National Academy of Sciences of the United States of America 107:15473-15478 (2010), Gong Y et al., PloS one 5: e12682 (2010)). Whether non-canonical Wnts share similar or different downstream signaling mechanisms is presently unclear.

Understanding the complexity of Wnt signaling is confounded by the intractable nature of Wnt proteins themselves; they are notoriously difficult to express, purify and maintain in a bioactive state. Wnts are modified post-translationally by glycosylation and acylation and have a tendency to be retained in the endoplasmic reticulum (reviewed by Coudreuse and Korswagen [Coudreuse D, Korswagen H C, Development 134:3-12 (2007)]). The addition of multiple lipid moieties renders them hydrophobic and likely contributes to their poor solubility and tendency to aggregate. Most functional studies to date have focused on mouse WNT3A and mouse WNT5A as representative examples of canonical and non-canonical Wnts, respectively. The substantial efforts studying these Wnts do not imply a lack of importance of the others, but rather reflect the fact that these were the first Wnts to be purified (Mikels A J, Nusse R, PLoS biology 4:e115 (2006), Willert K et al., Nature 423:448-452 (2003)) and that cell lines secreting them are available to researchers through the ATCC. While a number of recombinant Wnt proteins are commercially available, they are prohibitively expensive for many researchers and their quality and consistency have been questioned (Cajanek L et al., Journal of cellular biochemistry 111:1077-1079 (2010)). Furthermore, although purified protein may be ideal for acute studies, it is not practical for longer-term studies as Wnt proteins lose activity quickly in culture media, and periodic replenishment produces non-physiological activity spikes that do not model in vivo signaling processes. Transient transfection with Wnt expression vectors could mitigate the need for replenishment, but raises concerns about the consequences of heterogeneous expression levels and interpretation of phenotypes based on effects of supraphysiological Wnt levels in a small fraction of transfected cells.

Co-culture systems with cells that stably express Wnts provide a continuous source of active protein, resolving many of these issues. It follows that a system enabling regulatable production and secretion of bioactive Wnts would provide numerous experimental advantages. Although the commonly used mouse L cells and human HEK293 cells can produce bioactive Wnt proteins, neither is ideally suited for protein production. In contrast, CHO cells are the most widely used cell line for large-scale protein production in the biotechnology industry (Walsh G, Nature biotechnology 28:917-924 (2010)). CHO cells consistently produce a high yield of protein, can be grown at high-density under chemically defined conditions and are adaptable to suspension culture, thus permitting large scale production in bioreactors (reviewed by Wurm [Wurm F M, Nature biotechnology 22:1393-1398 (2004)]).

There is an unmet need in the art for an efficient mechanism to produce bioactive Wnt proteins for functional studies. The present invention cures these and other problems in the art.

### BRIEF SUMMARY OF THE INVENTION

Provided herein are, inter alia, efficient methods and compositions for expressing a protein (e.g. Wnt proteins) in a cell. Further provided herein is a cell expressing a protein (e.g. Wnt protein) and methods of isolating such protein in large quantities.

In one aspect, a method of expressing a protein in a cell is provided. The method includes (i) transfecting a stably transfected recombination cell with an expression nucleic acid and a recombinase nucleic acid, thereby forming an expression cell, wherein the expression nucleic acid includes a protein encoding nucleic acid sequence and a recombination selection nucleic acid. The method further includes (ii) allowing the expression cell to express the protein encoding nucleic acid, thereby expressing the protein.

In another aspect, a stably transfected recombination cell including an expression nucleic acid and a recombinase nucleic acid is provided. The expression nucleic acid includes a protein encoding nucleic acid sequence and a recombination selection nucleic acid.

In another aspect, an expression cell including a genome integrated expression nucleic acid is provided. The genome integrated protein expression nucleic acid includes a protein encoding nucleic acid sequence and a recombination selection nucleic acid.

### BRIEF DESCRIPTION OF THE DRAWINGS

FIG. 1. Generation of Wnt-expressing iCHO clones. FIG. 1A) Schematic of RMCE strategy. Parental CHO cells containing TetRKRAB, rtTA and a genomic acceptor cassette, located near the dihydrofolate reductase (DHFR) locus, were co-transfected with a plasmid containing the incoming exchange cassette and a plasmid encoding Cre recombinase. Upon expression of Cre, the L3 and 2L recognition sequences in the genome are recombined with the L3 and 2L recognition sequences in the incoming exchange cassette. This results in excision of HyTK, thus rescuing Ganciclovir sensitivity, and insertion of the Wnt-expression cassette, which confers Blasticidin resistance. FIG. 1B) Anti-FLAG western blot of cell lysates shows expression of FLAG-hWNT3A and FLAG-hWNT5A in three different iCHO clones. Protein loading was visualized by Ponceau staining (bottom). FIG. 1C) Cells were treated with 0, 0.1 or 1.0 μg/mL Dox. Anti-FLAG western blot of cell lysates shows expression of FLAG-hWNT3A and FLAG-hWNT5A. Near maximal expression was achieved with 0.1 μg/mL Dox. FIG. 1D) iCHO cells were grown in the presence of 0.25 μg/mL Dox for three days at which point conditioned media (CM) and cell lysates (L) were collected. Wnts and mFZ8CRD were immunoprecipitated from CM using anti-FLAG sepharose. While two species of FLAG-hWNT3A were visible in the cell lysate, only one form was visible in the CM, suggesting that the smaller species is not secreted.

FIG. 2. Activity of iCHO-produced Wnt Proteins. The graphs on the left depict induction of SUPERTOPFLASH (STF) activity in real-time bioluminescence monitoring assays. Values from the 24 hour timepoint were extracted for statistical analysis and are presented in the charts on the right. FIG. 2A) CM was collected from iCHO cells grown in the presence of Dox and 100 μL was added to reporter cells (mouse L+STF) in a 96 well plate. The volume of CM was kept constant such that “FLAG-hWNT3A:Par” contained 50 μL FLAG-hWNT3A CM and 50 μL CM from parental CHO cells. Thus, “FLAG-hWNT3A:FLAG-hWNT5A” CM and “FLAG-hWNT3A:Par” each have an equal amount of FLAG-hWNT3A CM. Arrow points to FLAG-hWNT1 in CM. FIG. 2B-2F) 293A-STF cells and iCHO cells were seeded together in a 96 well plate in the presence of Dox. Accumulation of luciferase activity is delayed compared to CM, presumably because it takes some time for the iCHO cells to produce Wnt following Dox stimulation. The total number of iCHO cells per well was kept constant such that “FLAG-hWNT3A” contained 100% FLAG-hWNT3A cells and “FLAG-hWNT3A:Par” contained 50% FLAG-hWNT3A cells and 50% Parental CHO cells.

FIG. 3. Different Wnt proteins display distinct activities. The graphs on the left depict induction of SUPERTOPFLASH (STF) activity in real-time bioluminescence monitoring assays. Values from the 24 hour timepoint were extracted for statistical analysis and are presented in the charts on the right. FIG. 3A-3C) 293A-STF cells and CHO cells were seeded together in a 96 well plate in the presence of Dox, the total number of CHO cells per well was kept constant.

FIG. 4. WNT3A has a greater range of activity than WNT1. FIG. 4A) Western blot detecting the FLAG epitope comparing the amount of hWNT1 v. hWNT3A in cell lysates and conditioned media. FIG. 4B) Flow cytometry analysis detecting the FLAG epitope comparing the amount of different WNTs on the surface of live iCHO cells following Dox induction. FIG. 4C) iCHO cells were co-cultured with 293A-STF cells at varying cell densities. The ratio of hWNT3A-induced STF activity to hWNT1-induced STF activity is shown for three timepoints.

FIG. 5. Optimized RMCE selection strategy. FIG. 5A) Schematic of positive and negative selection strategy to reduce the emergence of false-positive clones. FIG. 5B) Flow cytometry analysis of RMCE efficiency. Parental CHO cells (CHO111-134) were engineered by RMCE with a mCitrine expression cassette using the positive and negative selection strategy. Four pools of clones were analyzed, each of which had greater than 99% mCitrine positive cells.

FIG. 6. Wnt production, secretion and stability. FIG. 6A) Anti-WNT3A and anti-WNT5A western blots comparing the levels of tagged and untagged WNTs in cell lysates and conditioned media. FIG. 6B) Anti-WNT3A western blots comparing the levels of mWNT3A produced by L cells or iCHO cells in cell lysates and conditioned media. FIG. 6C) Coomassie stain of purified hWNT3A and mWNT3A separated by SDS-PAGE. FIG. 6D) 293A-STF cells were treated with 200 ng/mL purified hWNT3A or mWnt3A protein that was either fresh from 4° C., or pre-incubated at 37° C. for 6 hr or 24 hr.

FIG. 7. Activity of hWNT5A purified from iCHO cells. FIG. 7A) Coomassie stain of purified hWNT5A. FIG. 7B) 293-STF reporter assay showing that hWNT5A on its own doesn't activate STF, but is able to inhibit WNT3A activation of the pathway.

FIG. 8. Flow cytometry analysis of surface FLAG-WNTs. iCHO cells were induced with 250 ng/mL Dox and harvested after 48 hours with either 50 mM EDTA or 0.05% trypsin. They were then stained with anti-FLAG (M2) primary antibody and anti-mouse AF568 secondary antibody. Live cells (DAPI negative) are shown in the plots above. The top three panels are negative controls. iCHO WNT1 cells react strongly when harvested with EDTA, but the signal is decreased when the cells are harvested by trypsin.

### DETAILED DESCRIPTION OF THE INVENTION

### I. Definitions

Unless defined otherwise, technical and scientific terms used herein have the same meaning as commonly understood by a person of ordinary skill in the art. See, e.g., Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The phrase “stringent hybridization conditions” refers to conditions under which a probe will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, TECHNIQUES IN BIOCHEMISTRY AND MOLECULAR BIOLOGY—HYBRIDIZATION WITH NUCLEIC PROBES, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

A variety of methods of specific DNA and RNA measurement that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST/ or the like. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “transfection” or “transfected” are defined by a process of introducing nucleic acid molecules into a cell by non-viral and viral-based methods. The nucleic acid molecules may be gene sequences encoding complete proteins or functional portions thereof. For non-viral methods of transfection any appropriate transfection method that does not use viral DNA or viral particles as a delivery system to introduce the nucleic acid molecule into the cell is useful in the methods described herein. Exemplary transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using retroviral vectors. In other embodiments, the nucleic acid molecules are introduced into a cell using lentiviral vectors.

The term “isolated,” when applied to a protein, denotes that the protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a protein gives rise to essentially one band in an electrophoretic gel. Particularly, it means that the protein is at least 85% pure, more preferably at least 95% pure, and most preferably at least 99% pure.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. In some embodiments, the transfected gene forms part of the genome of the transfected cell. Where the transfected gene forms part of the genome of the transfected cell the gene is integrated in the cellular chromosome of the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

A “Wnt protein” as referred to herein includes any of the naturally-occurring forms of the Wnt signaling factor, or variants thereof that maintain Wnt signaling factor activity (e.g. within at least 50%, 80%, 90% or 100% activity compared to Wnt). In some embodiments, variants have at least 90% amino acid sequence identity across their whole sequence compared to the naturally occurring Wnt polypeptide. In some embodiments, the Wnt polypeptide is encoded by the nucleic acid sequence of SEQ ID:1, SEQ ID:2, SEQ ID:3, SEQ ID:4, SEQ ID:5, or SEQ ID:6.

### II. Methods of Producing Proteins in a Cell

Provided herein are methods of producing a protein in a cell. In more particular, methods of expressing a human protein (e.g. a Wnt protein) in a cell are provided. In one aspect, a method of expressing a protein in a cell is provided. The method includes (i) transfecting a stably transfected recombination cell with an expression nucleic acid and a recombinase nucleic acid, thereby forming an expression cell, wherein the expression nucleic acid includes a protein encoding nucleic acid sequence and a recombination selection nucleic acid. The method further includes (ii) allowing the expression cell to express the protein encoding nucleic acid, thereby expressing the protein.

A “stably transfected recombination cell” as provided herein is a cell that includes a recombination donor site. The recombination donor site is a nucleic acid encoding a recombinase recognition sequence, a positive selection protein and a negative selection protein. Recombinase recognition sequences are nucleic acid sequences, which are recognized by recombinase enzymes (e.g. Cre recombinase). The use of recombinase enzymes and their recognition sites to excise and or/replace specific nucleic acid sequences from their site of integration is referred to as “recombination” and is well known in the art. See for example: Nagy A (2000). “Cre Recombinase: The Universal Reagent for Genome Tailoring”. Genesis 26: 99-109; Sternberg & Hamilton (1981). “Bacteriophage P1 site-specific recombination:1. Recombination between loxP sites”. Journal of Molecular Biology 150: 467-486. In some embodiments, the recombination donor site is part of (i.e. is integrated into) the genome of the stably transfected recombination cell. Where the recombination donor site is part of the genome of the stably transfected recombination cell, the nucleic acid encoding a recombinase recognition sequence, a positive selection protein and a negative selection protein is integrated into the genome of the stably transfected recombination cell. The recombinase recognition sequence may be operably linked to the nucleic acid encoding a positive selection protein and a negative selection protein, to provide for excision of the nucleic acid encoding a positive selection protein and a negative selection protein from the genome of the stably transfected recombination cell in the presence of a recombinase enzyme. In some embodiments, the recombinase recognition sequence forms a first recombinase recognition sequence and a second recombinase recognition sequence, wherein the first recombinase recognition sequence flanks the 5′ end of the nucleic acid encoding a positive selection protein and a negative selection protein and the second recombinase recognition sequence flanks the 3′ end of the nucleic acid encoding a positive selection protein and a negative selection protein.

A positive selection marker is a nucleic acid sequence encoding a protein (“positive selection protein”) that confers resistance toward a cellular toxin. In some embodiments, the positive selection protein is a polypeptide with enzymatic activity. The enzymatic activity includes, but is not limited to, the activity of an acetyltransferase and a phosphotransferase. In some embodiments, the enzymatic activity of the positive selection protein is the activity of a phosphotransferase. The enzymatic activity of the positive selection protein may confer a stably transfected recombination cell the ability to expand in the presence of a toxin. A toxin is a compound capable of inhibiting cell expansion and/or causes cell death. Examples of such toxins include, but are not limited to hygromycin, neomycin, puromycin and gentamycin. In some embodiments, the toxin is hygromycin. Through the enzymatic activity of a positive selection protein a toxin may be converted to a non-toxin, which no longer inhibits expansion and causing cell death of a stably transfected recombination cell. Upon exposure to a toxin a cell lacking a positive selection maker may be eliminated and thereby precluded from expansion.

A negative selection marker is a nucleic acid sequence encoding a protein (“negative selection protein”) that confers sensitivity toward a negative selection substrate. In some embodiments, the negative selection protein is a polypeptide with enzymatic activity. The enzymatic activity includes, but is not limited to, the activity of a phosphotransferase. In some embodiments, the enzymatic activity of the negative selection protein is the activity of a phosphotransferase. In some further embodiments, the phosphotransferase is thymidine kinase. The enzymatic activity of the negative selection protein confers to a stably transfected recombination cell the ability to expand in the absence of a negative selection substrate. However, in the presence of a negative selection substrate cell expansion is inhibited and/or the stably transfected recombination cell dies. Examples of such substrates include, but are not limited to ganciclovir, acyclovir, and idoxuridine. In some embodiments, the negative selection substrate is ganciclovir. Through the enzymatic activity of the negative selection protein a negative selection substrate is converted (e.g. phosphorylated) to a toxin, which inhibits expansion and causes cell death of a stably transfected recombination cell. In the absence of a negative selection substrate growth and viability of the stably transfected recombination cell remain uncompromised.

In some embodiments the stably transfected recombination cell is a mammalian cell. In some further embodiments, the stably transfected recombination cell is an adherent cell. An adherent cell as described herein is a cell, which upon cultivation (i.e. expansion) grows attached to the surface of a cell cultivation container. In contrast, a non-adherent cell does not grow attached to the surface of a cell cultivation container, but upon expansion forms a suspension of cells in the cultivation media. In some embodiments, the stably transfected recombination cell is a CHO (Chinese Hamster Ovary) cell. A non-limiting example of a stably transfected recombination cell is described by Wong et al. 2005, which is hereby incorporated by reference in its entirety and for all purposes.

For the methods provided herein a stably transfected recombination cell may be transfected with an expression nucleic acid and a recombinase nucleic acid to form an expression cell. The recombinase nucleic acid is a nucleic acid that encodes a recombinase enzyme (e.g. Cre recombinase). In some embodiments, the recombinase nucleic acid forms part of a plasmid. In other embodiments, the recombinase nucleic acid forms part of a viral vector. Upon expression in a stably transfected recombination cell and in the presence of a recombinase recognition sequence the recombinase enzyme provides for recombination. As described above recombination is a cellular process well known in the art. Recombination as provided herein is the process of removing from the genome of a stably transfected recombination cell the positive selection marker (i.e. nucleic acid encoding the positive selection protein) and the negative selection marker (i.e. nucleic acid encoding the negative selection protein) and replacing it with an expression nucleic acid, thereby forming an expression cell. An expression cell as referred to herein is a cell which does not express a negative selection protein. Thus, an expression cell does not exhibit sensitivity towards a negative selection substrate (e.g. ganciclovir). Therefore, in the presence of a negative selection substrate an expression cell (i.e. a cell that has undergone the process of recombination) is able to grow, whereas a stably transfected recombination cell (i.e. a cell which expresses the negative selection protein) is not able to grow. In some embodiments, the negative selection substrate is ganciclovir.

An “expression nucleic acid” as provided herein is a nucleic acid including a protein encoding nucleic acid sequence to be expressed. Typically, all elements necessary to express (e.g. transcription regulatory sequences, translation regulatory sequences) a protein (e.g. Wnt protein, Wnt protein antagonist) in a cell is present on the expression nucleic acid. As provided herein the expression nucleic acid may include a protein encoding nucleic acid sequence (e.g. Wnt protein encoding nucleic acid sequence) and a recombination selection marker. The recombination selection marker may be a positive selection marker (e.g. such as those described above). A protein encoding nucleic acid sequence as referred to herein may include the coding region sequence (i.e. cDNA) of a protein of interest (e.g. Wnt protein), a fragment, a variant or a functional equivalent thereof. In some embodiments, the protein is a Wnt protein and the protein encoding nucleic acid sequence is a Wnt protein encoding nucleic acid sequence. In other embodiments, the Wnt protein encoding nucleic acid sequence includes SEQ ID:1, SEQ ID:2, SEQ ID:3, SEQ ID:4, SEQ ID:5 or SEQ ID:6. In some embodiments, the Wnt protein encoding nucleic acid sequence includes SEQ ID:1. In some embodiments, the Wnt protein encoding nucleic acid sequence includes SEQ ID:2. In some embodiments, the Wnt protein encoding nucleic acid sequence includes SEQ ID:3. In some embodiments, the Wnt protein encoding nucleic acid sequence includes SEQ ID:4. In some embodiments, the Wnt protein encoding nucleic acid sequence includes SEQ ID:5. In some embodiments, the Wnt protein encoding nucleic acid sequence includes SEQ ID:6. In other embodiments, the protein is a Wnt protein antagonist (e.g. FZD8CRD) and the protein encoding nucleic acid sequence is a Wnt protein antagonist encoding nucleic acid sequence.

For the methods provided herein, the expression nucleic acid may further include a promoter nucleic acid sequence controlling expression of the protein encoding nucleic acid sequence. Thus in some embodiments, the expression nucleic acid may further include a promoter nucleic acid sequence operably linked to the protein encoding nucleic acid sequence. The promoter nucleic acid may be a constitutively active promoter. Alternatively, the promoter nucleic acid sequence may be an inducible promoter. Inducible promoters are regulatory nucleic acids whose activity is triggered by either chemical or physical factors. Chemically induced promoters are promoters whose transcriptional activity is regulated by the presence or absence of organic or inorganic compounds (i.e. promoter inducing compounds). Non-limiting examples of organic or inorganic compounds regulating inducible promoters are alcohol, antibiotics (e.g. doxycycline, tetracycline) steroids, or metals. Physically induced promoters are promoters whose transcriptional activity is regulated by the presence or absence of light and low or high temperatures. In some embodiments, the promoter nucleic acid sequence is an inducible promoter nucleic acid sequence. In some embodiments, the promoter nucleic acid sequence is a doxycycline-inducible promoter nucleic acid sequence. In some further embodiments, the protein encoding nuclei acid sequence is a Wnt protein encoding nucleic acid sequence.

The expression nucleic acid used according to the methods provided herein may include a recombination selection nucleic acid (i.e. recombination selection marker). The terms “recombination selection nucleic acid” and “recombination selection marker” are used interchangeably throughout this disclosure. A recombination selection marker as provided herein refers to a nucleic acid encoding a protein (“recombination selection protein”) that confers a selection advantage. The recombination selection protein may have enzymatic activity. The enzymatic activity includes, but is not limited to, the activity of an acetyltransferase and a phosphotransferase. In some embodiments, the enzymatic activity of the recombination selection protein is the activity of a phosphotransferase. The enzymatic activity of the recombination selection protein may confer an expression cell the ability to expand in the presence of a recombination selection substrate. A recombination selection substrate is a compound capable of inhibiting cell expansion and/or causing cell death of an expression cell. Examples of recombination selection substrates include, but are not limited to blasticidin, hygromycin, neomycin, puromycin and gentamycin. In some embodiments, the recombination selection substrate is blasticidin. Through the enzymatic activity of a recombination selection protein a recombination selection substrate may be converted to a non-toxic compound, which no longer inhibits expansion and/or causes cell death of an expression cell. Upon exposure with a recombination selection substrate a cell lacking a recombination selection protein may be eliminated and thereby precluded from expansion.

According to the methods provided herein including embodiments thereof, a stably transfected recombination cell may be transfected with an expression nucleic acid and a recombinase nucleic acid, thereby forming an expression cell. In some embodiments, the expression nucleic acid forms part of a first nucleic acid and the recombinase nucleic acid forms part of a second nucleic acid. In other embodiments, the expression nucleic acid and the recombinase nucleic acid form part of the same nucleic acid. An expression cell provided herein is formed by recombination as described above. The recombination donor site is excised from the genome of the stably transfected recombination cell (excision step) and replaced with the expression nucleic acid by integration of the expression nucleic acid sequence into the same location within the genome (integration step). In order to select for cells that have completed the excision step, a negative selection substrate (e.g. ganciclovir) is administered to the cells upon transfection with the expression nucleic acid and the recombinase nucleic acid. In some embodiments, the negative selection substrate is administered after transfection. In some further embodiments the negative selection substrate is administered less than 48 hours after (e.g. approximately 12 hours, 24 hours, or 36 hours after the transfection). Further, to select for cells that have completed both the excision step and the integration step, a recombination selection substrate is administered. The negative selection substrate and the recombination selection substrate may be administered subsequently. In some embodiments, the negative selection substrate and the recombination selection substrate are administered simultaneously (i.e. at the same time). In some embodiments, the recombination selection substrate is administered approximately 3 to 6 days (e.g. 3, 4, or 5 days) after transfection. In other embodiments, the recombination selection substrate is administered approximately 2 to 5 days (e.g. 2, 3, or 4 days) after administering the negative selection substrate. Thus, in some embodiments, the method further includes after the transfecting of step (i) and before the allowing of step (ii), a step (i.a) of administering to the expression cell a negative selection substrate (e.g. ganciclovir). In some further embodiments, the negative selection substrate is ganciclovir. In some embodiments, the transfecting of step (i) further includes administering a negative selection substrate to the stably transfected recombination cell. In other embodiments, the method further includes after the administering of (i.a), a step (i.b) of administering a recombination selection substrate. In some further embodiments, the recombination selection substrate is blasticidin.

As mentioned above the expression nucleic acid may further include an inducible promoter nucleic acid sequence operably linked to the protein encoding nucleic acid sequence. For the inducible promoter nucleic acid sequence to be activated, a promoter inducing compound may be administered. The promoter inducing compound (e.g. doxycycline) may be administered to the expression cell at the same time as the recombination selection substrate (e.g. blasticidin). Thus, in some embodiments, administering of (i.b) further includes administering a promoter inducing compound. In some further embodiments, the promoter inducing compound is doxycycline.

For the methods provided herein the expression cell is allowed to express the protein encoding nucleic acid, thereby expressing the protein. The “allowing to express” includes expansion of the expression cell after transfection, optional selection for transfected cells and identification of expression cells. Expansion as used herein includes the production of progeny cells by a transfected stably transfected recombination cell in containers and under conditions well known in the art. Expansion may occur in the presence of suitable media and cellular growth factors. Cellular growth factors are agents which cause cells to migrate, differentiate, transform or mature and divide. They are polypeptides which can usually be isolated from various normal and malignant mammalian cell types. Some growth factors can also be produced by genetically engineered microorganisms, such as bacteria (E. coli) and yeasts. Cellular growth factors may be supplemented to the media. Examples of cellular growth factors include, but are not limited to, SCF, GM-CSF, FGF, bFGF2, and EGF.

In some embodiments, the method provided herein further includes separating the Wnt protein from the expression cell, thereby preparing a free Wnt protein. A free Wnt protein is a protein free of other cellular components with which it is associated in the natural state. A free Wnt protein resides outside of the cell by which it is produced. A free Wnt protein may be secreted by the cell by which it is produced. Thus in some further embodiments, the free Wnt protein is a secreted Wnt protein. In other embodiments, the free Wnt protein is an intracellular Wnt protein. An intracellular Wnt protein resides inside of the cell by which it is produced and may be associated with subcellular structures (e.g. cellular membranes, organelles).

### III. Compositions

Provided herein are cells useful in the production (e.g. expression) of proteins (e.g. Wnt proteins). A person of skill will immediately recognize that the terms described above are applicable to the following paragraphs.

In another aspect, a stably transfected recombination cell (e.g. a mammalian cell) including an expression nucleic acid and a recombinase nucleic acid is provided. The expression nucleic acid includes a protein encoding nucleic acid sequence (e.g. a Wnt protein encoding sequence) and a recombination selection nucleic acid. The recombination selection nucleic acid may as described above encode a recombination selection protein. In some embodiments, the expression nucleic acid further includes a promoter nucleic acid sequence operably linked to the protein encoding nucleic acid sequence. In other embodiments, the promoter nucleic acid sequence encodes an inducible promoter as described above.

In another aspect, an expression cell including a genome integrated expression nucleic acid is provided. The genome integrated expression nucleic acid includes a protein encoding nucleic acid sequence and a recombination selection nucleic acid. In some embodiments, the expression cell is derived from a stably transfected recombination cell. In other embodiments, the expression nucleic acid further includes a promoter nucleic acid sequence operably linked to the protein encoding nucleic acid sequence. In some embodiments, the protein encoding nucleic acid sequence is a Wnt protein encoding nucleic acid sequence. In other embodiments, the Wnt protein encoding nucleic acid sequence is the sequence of SEQ ID:1, SEQ ID:2, SEQ ID:3, SEQ ID:4, SEQ ID:5, or SEQ ID:6.

### IV. Examples

Applicants describe a system that incorporates a Cre recombinase mediated cassette exchange (RMCE) and inducible transgene expression that Applicants used to generate eleven new CHO cell lines expressing tagged and un-tagged human Wnts or the Wnt antagonist FZD8CRD. Applicants used Cre to deliver the Wnt expression transgenes into a floxed locus downstream of the DHFR locus (Wong E T et al., Nucleic acids research 33:e147 (2005)). This strategy ensures that the integrated Wnt transgene is present in a defined locus, enabling reproducible and consistent expression of different Wnt proteins between independently derived cell lines. Furthermore, Wnt expression is controlled by Doxycycline (Dox), thus providing tunable expression levels and a “minus Dox” condition for a negative control. Applicants have purified both WNT3A and WNT5A proteins from media conditioned by these inducible CHO (iCHO) cells and found that these proteins are active in novel real-time bioluminescence Wnt reporter assays. These cell lines should make functional studies of human Wnt systems more accessible, reliable and reproducible in research laboratories and reduce the cost of making and purifying Wnt proteins

### Example 1

### Engineering Wnt-Producing iCHO Cell Lines

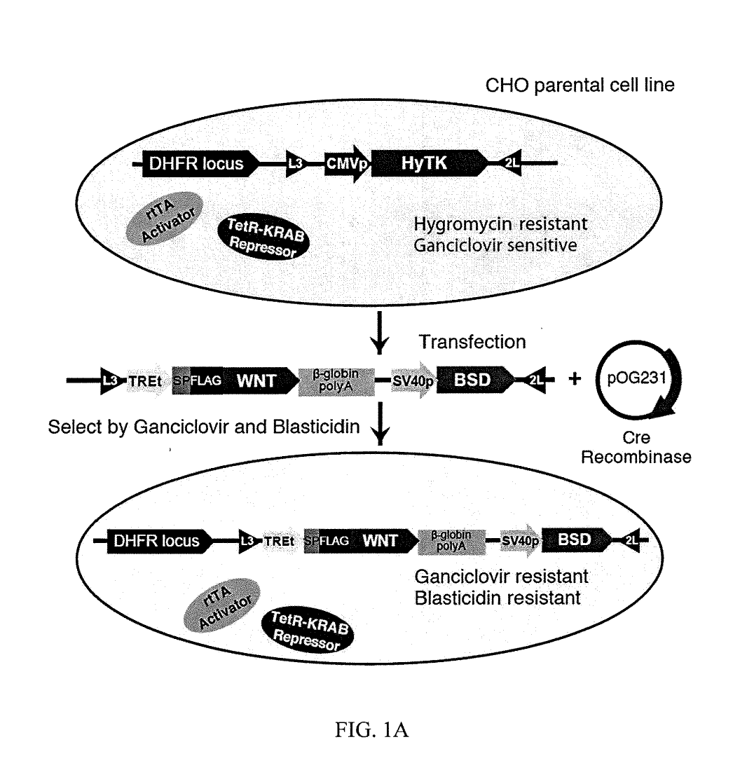

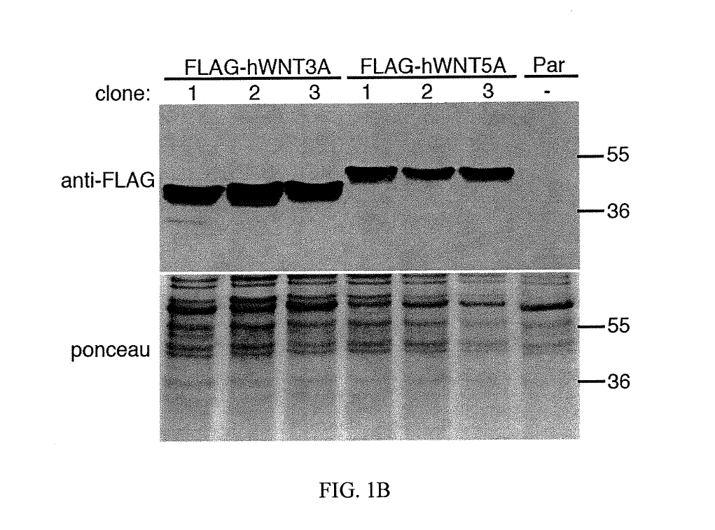

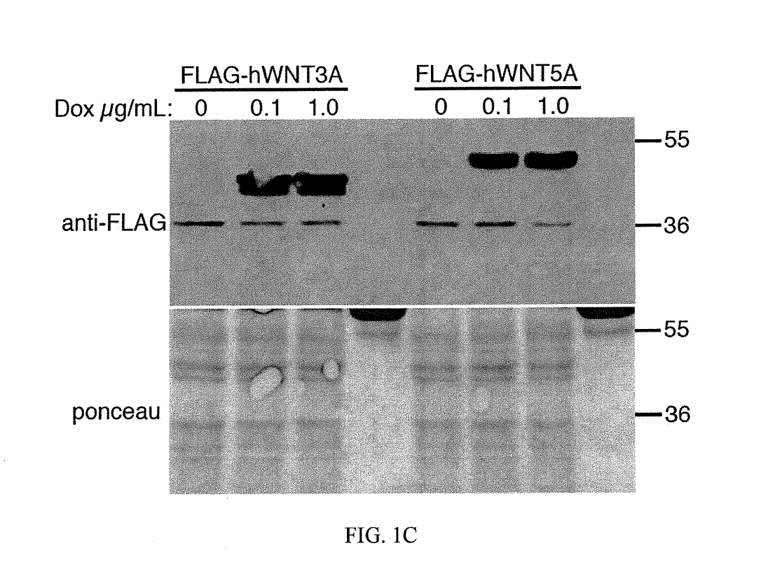

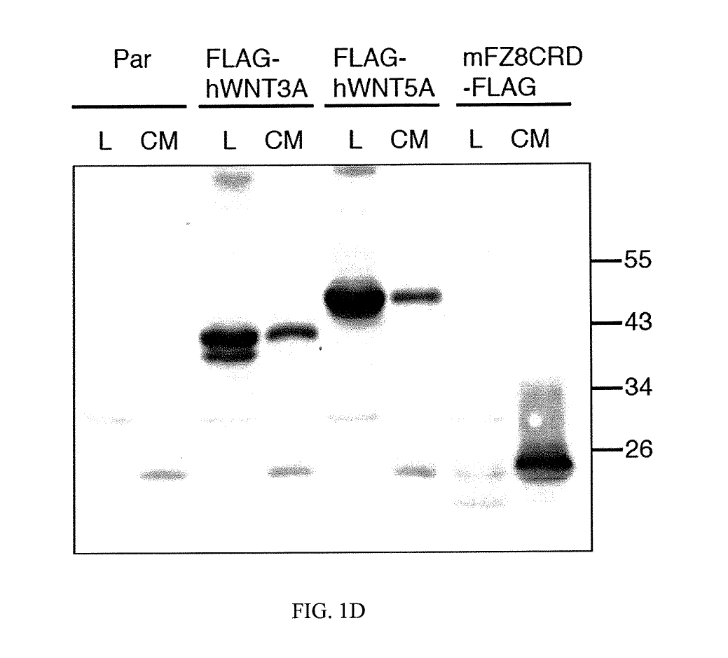

Applicants previously described a Dox-controlled transgene expression system in mammalian cells in which a transgene can be efficiently targeted to a genomic locus (Wong E T et al., Nucleic acids research 33:e147 (2005)). The locus was selected to exhibit low basal expression, and to confer tight Dox-inducible transgene expression. The parental CHO cell line contains a genomic acceptor cassette comprised of HyTK, which confers Hygromycin resistance and Ganciclovir sensitivity, flanked by the heterologous LoxP sites L3 and 2L. The parental line also contains the reverse tetracycline transactivator (rtTA) and the TetR-KRAB repressor integrated at a separate genomic location to confer Dox inducibility with minimal expression in the absence of Dox (FIG. 1A). Importantly, the integrated transgene exhibits reproducible levels of Dox-dependent induction in independently-derived clones.

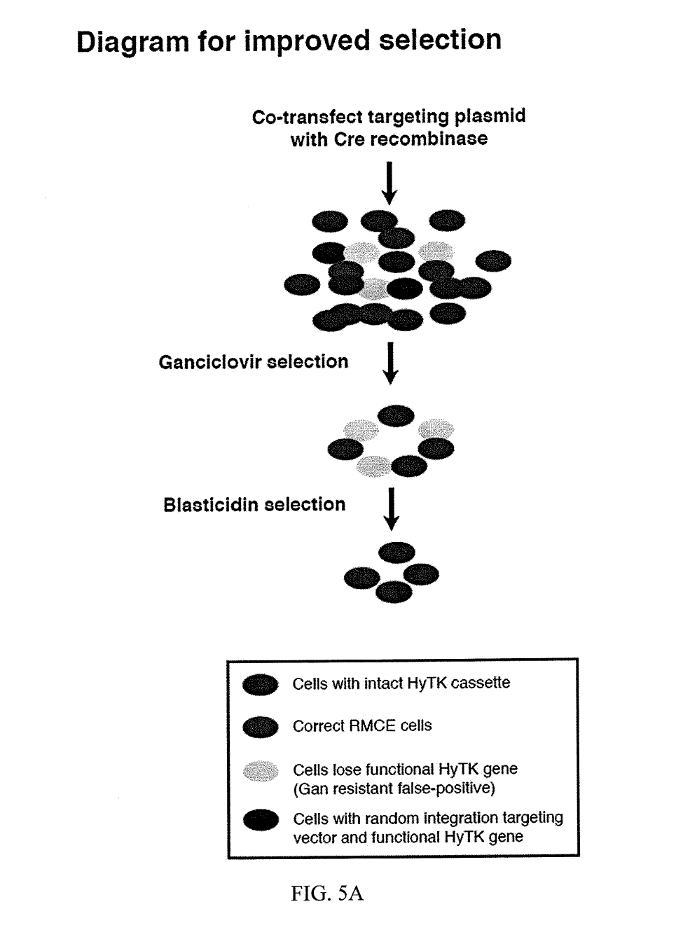

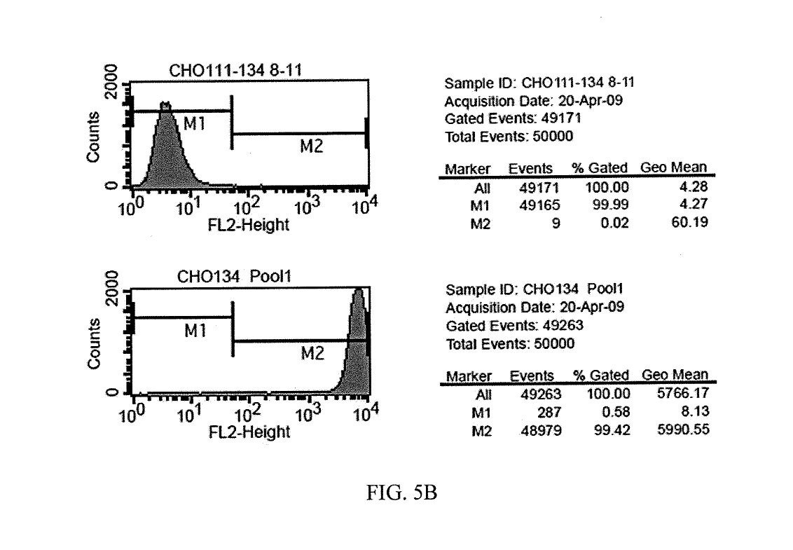

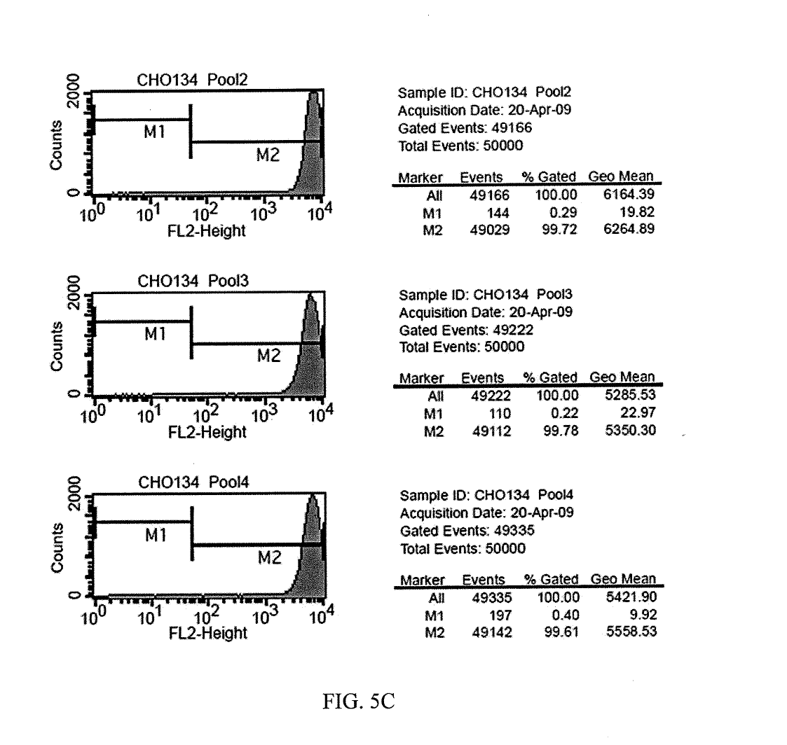

The average RMCE efficiency in these CHO cells was extremely high, with 80% of drug selected clones carrying a site-specific insertion (Wong E T et al., Nucleic acids research 33:e147 (2005)). More recently, Applicants have found that false-positive RMCE clones can result from either loss of the HyTK cassette, or silencing of the TK gene (data not shown), both of which confer Ganciclovir resistance. Applicants developed the following strategy to minimize emergence of non-RMCE induced Ganciclovir resistant clones. Applicants introduced a Blasticidin drug resistance gene (BSD) into the donor exchange vector and optimized the RMCE selection scheme with two rounds of drug selection: first, negative selection with Ganciclovir selects for the absence of the HyTK cassette; second, positive selection with Blasticidin selects for the presence of the transgene cassette (FIG. 5A). This improved RMCE selection strategy results in the generation of correctly targeted clones with greater than 99% fidelity (FIG. 5B).

To express human Wnt proteins, the incoming exchange vector contains human Wnt cDNA regulated by the Dox-inducible TRE-tight promoter. Applicants engineered a single FLAG tag at the N-terminus of several human Wnt proteins, including hWNT3A and hWNTSA, immediately following the hWNT3A signal sequence to enable comparison of the expression levels of different Wnts. The soluble Wnt inhibitor mFZD8CRD (Hsieh J C et al., Proceedings of the National Academy of Sciences of the United States of America 96:3546-3551 (1999); Reya T et al., Nature 423:409-414 (2003)) contains a FLAG tag at the C-terminus. The incoming exchange vector was transfected into the parental CHO cell line along with a Cre-recombinase expression vector. After sequential rounds of selection with Ganciclovir and Blasticidin, clones were isolated and transgene expression was confirmed by immune-blot (FIG. 1B). Using this strategy, Applicants rapidly generated multiple clones of inducible CHO lines (iCHO) that displayed undetectable background Wnt expression and similar levels of Wnt expression following Dox induction (FIG. 1C). Anti-FLAG immunoprecipitation confirmed the presence of WNT3A, WNT5A and mFZD8CRD in conditioned media (FIG. 1D).

### Example 2

### iCHO Cells Secrete Active Human Wnt Proteins in a Regulatable Manner

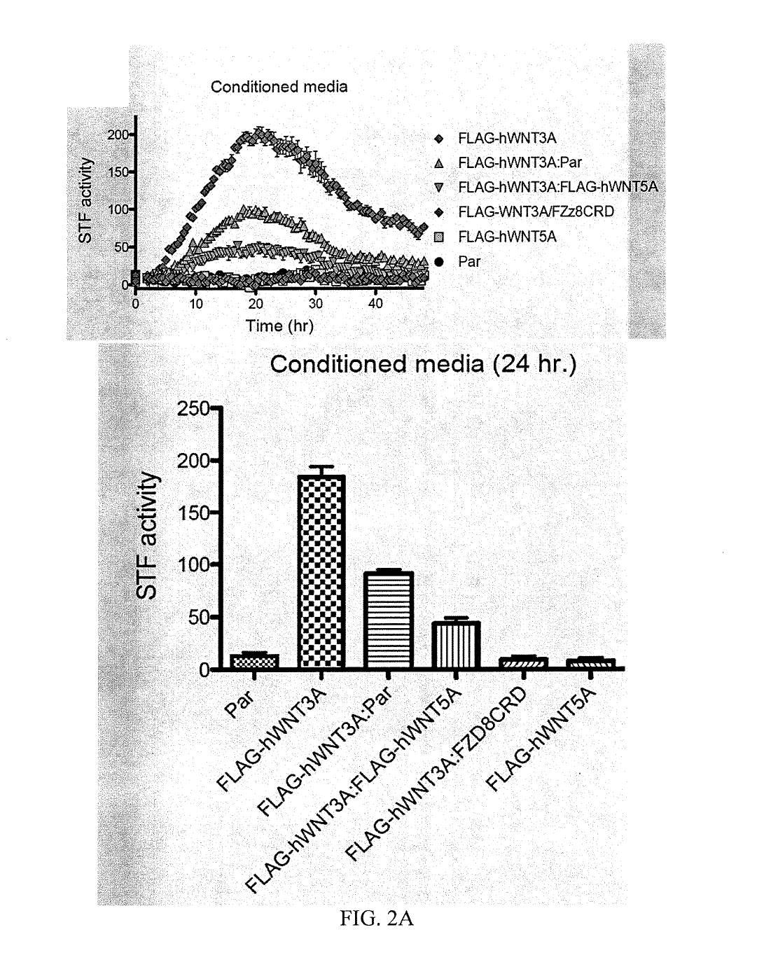

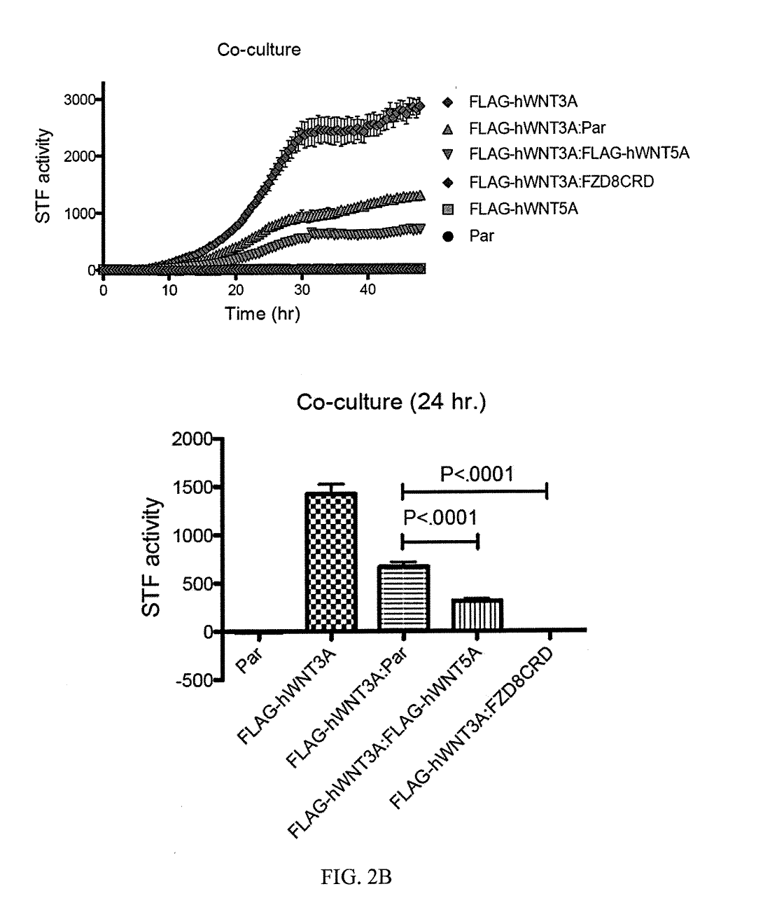

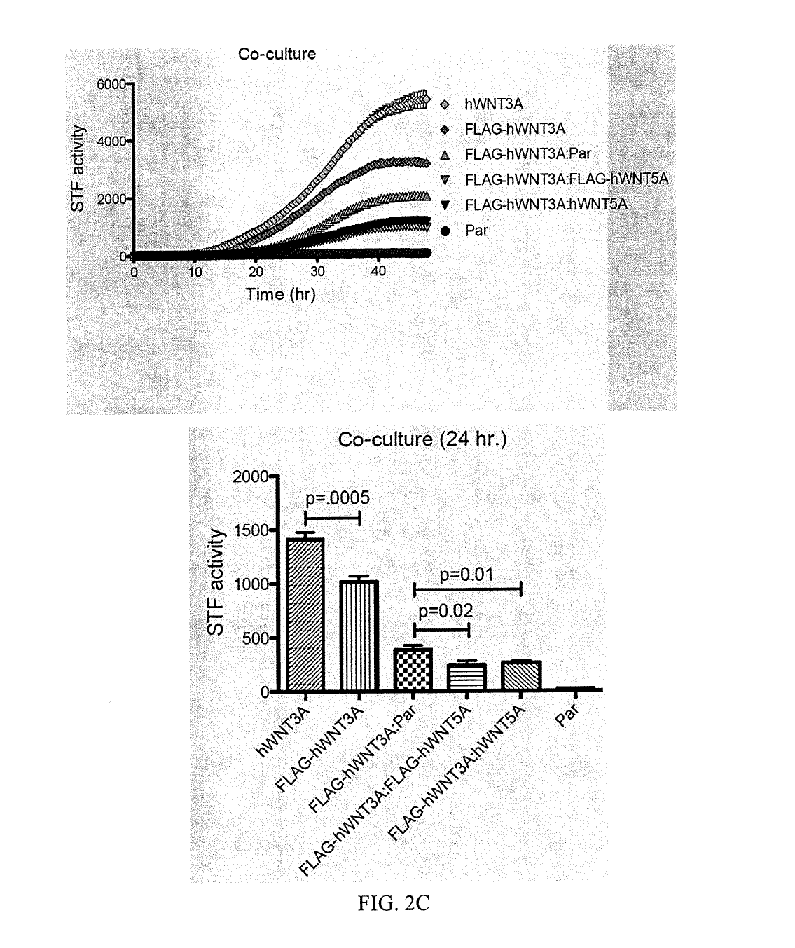

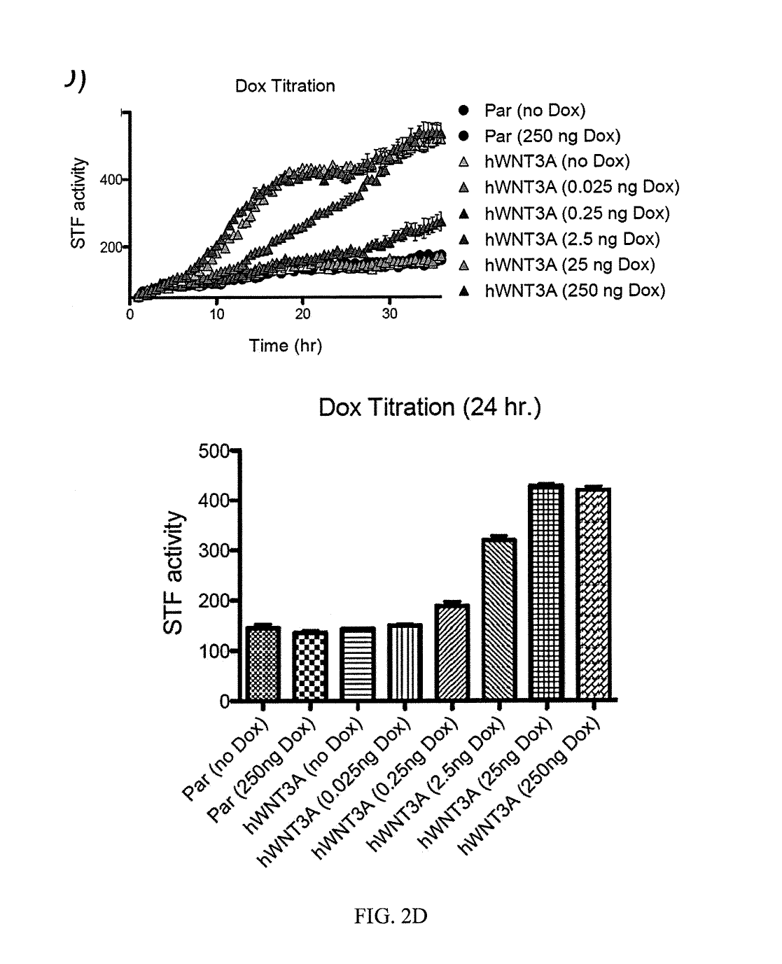

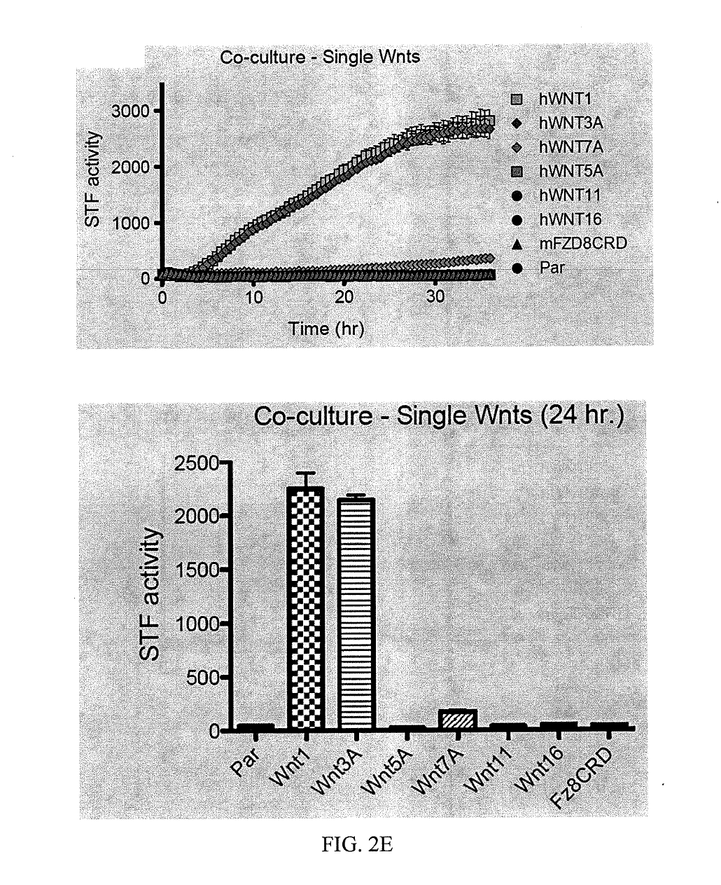

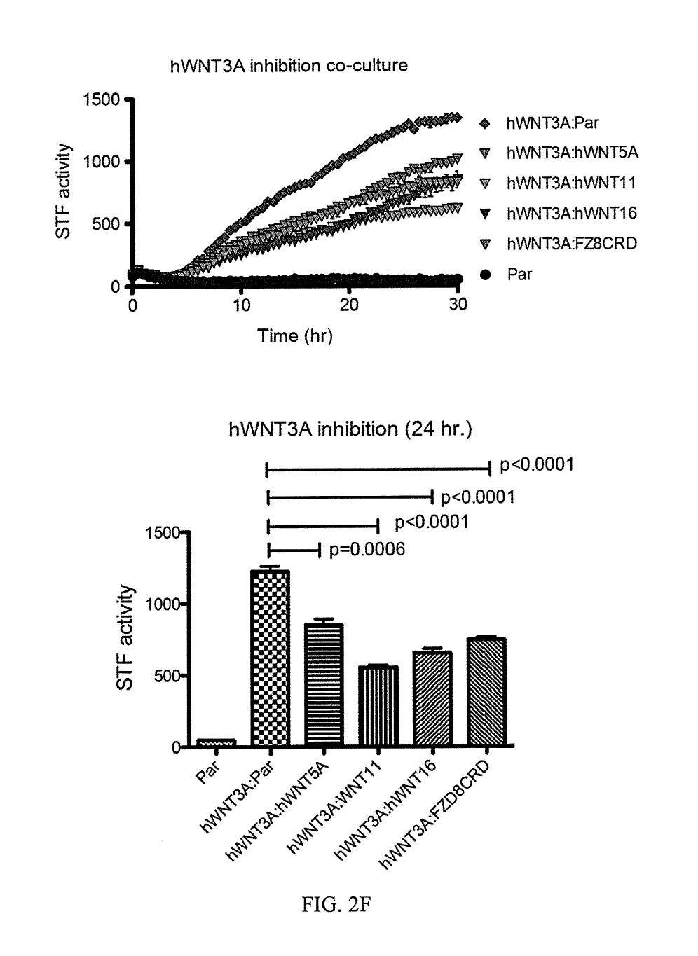

The Super-TOPFLASH (STF) luciferase reporter assay is a well-established indicator of canonical Wnt activity (Korinek V et al., Science 275:1784-1787 (1997), Veeman M T et al., Current biology: CB 13:680-685 (2003b)). Non-canonical WNT5A activity can be measured by inhibition of WNT3A-induced STF activity (Mikels A J, Nusse R, PLoS biology 4:e115 (2006)). Using this reporter, Applicants evaluated the activity of iCHO-produced Wnts by real-time bioluminescence monitoring assays (Pulivarthy S R et al., Proceedings of the National Academy of Sciences of the United States of America 104:20356-20361 (2007)), whereby STF luciferase activity was measured in live cells over 24-48 hours. Treatment of mouse L cells that stably carry the STF reporter (Mikels A J, Nusse R, PLoS biology 4:e115 (2006)) with iCHO-produced FLAG-hWNT3A conditioned media resulted in robust induction of luciferase activity that peaked around 20 hours post treatment (FIG. 2A). This activity was inhibited ˜50% by FLAG-hWNT5A and ˜100% by mFZD8CRD-FLAG conditioned media, indicating that iCHO cells secrete Wnts with the expected activities. In co-culture experiments, where iCHO cells and 293A-STF reporter cells were grown in the same well, Wnt activity was more sustained compared to stimulation by conditioned media (FIG. 2B), suggesting that Wnts in the conditioned media become depleted or inactivated over time (see below).

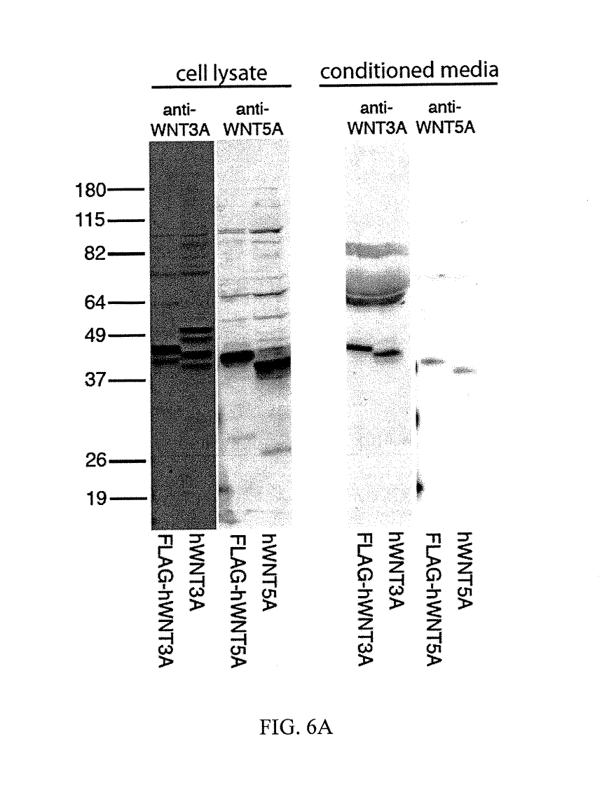

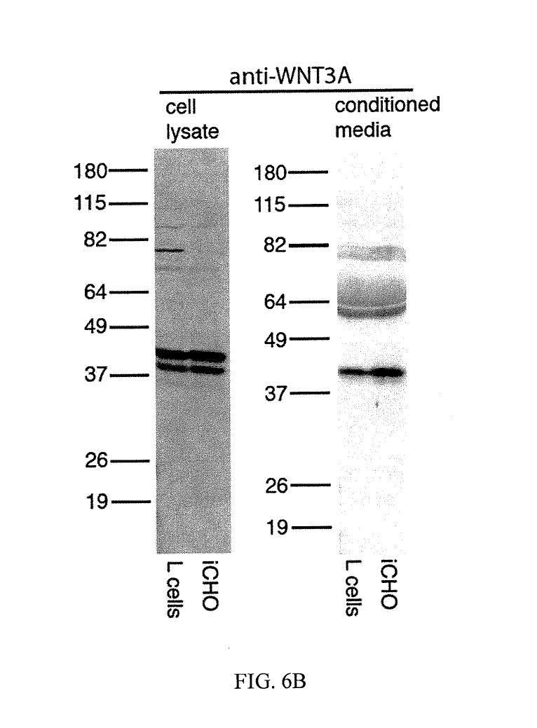

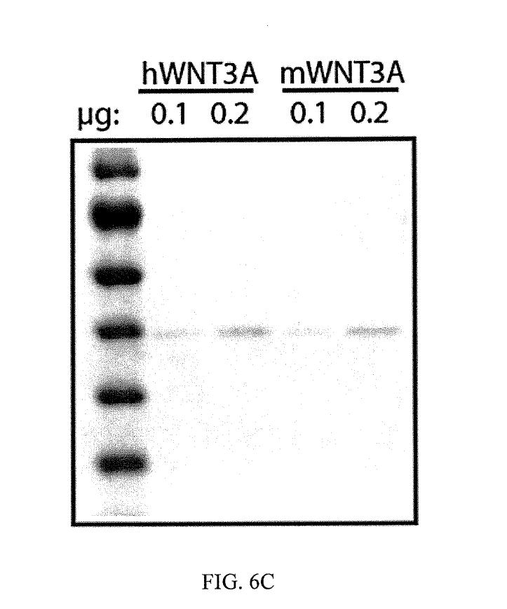

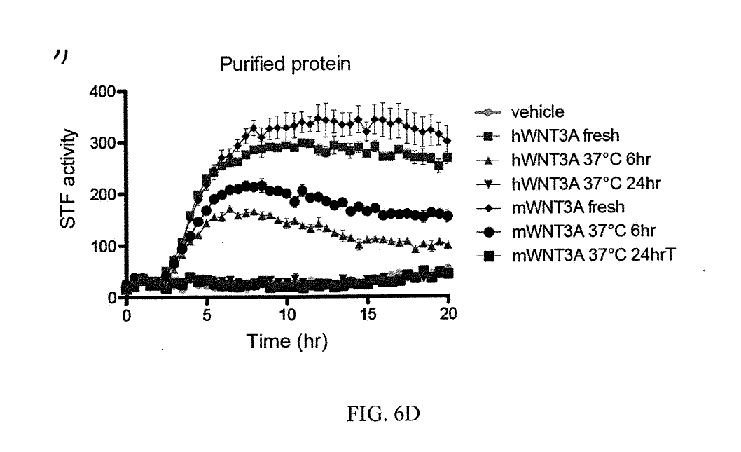

Applicants next generated iCHO cell lines expressing untagged versions of hWNT3A and hWNT5A because it was possible that the FLAG tag altered Wnt activity. iCHO-hWNT3A cells induced a greater WNT response than iCHO-FLAG-hWNT3A cells in co-culture with 293A-STF cells (FIG. 2C). The FLAG tag did not reduce the expression or secretion of hWNT3A (FIG. 6A), indicating that the FLAG tag reduces the activity of hWNT3A protein. In contrast, FLAG-hWNT5A and hWNT5A behaved similarly, as measured by antagonism of hWNT3A activity (FIG. 2C) and were similarly expressed and secreted.

As Wnts are classic morphogens that exert their effects on responding cells in a concentration dependent manner (reviewed by Ashe and Briscoe [Ashe H L, Briscoe J, Development 133:385-394 (2006)]), an optimal system would enable modulation of Wnt expression levels. Applicants therefore assessed the inducibility of hWNT3A expression by treating iCHO-hWNT3A cells with varying Dox concentrations and measuring induction of the Wnt-dependent reporter in co-culture with 293A-STF cells. The results clearly show a Dox-dependent induction of Wnt reporter activity in this co-culture system (FIG. 2D). In agreement with western blot analysis (see FIG. 1C), iCHO-hWNT3A in the absence of Dox and parental CHO cells induced virtually the same luminescence levels when mixed with the Wnt-responsive 293-STF cells, indicating that this inducible system has little if any expression in the absence of inducer, and a very high signal to noise ratio upon Dox addition (FIG. 2D).

Non-canonical or beta-catenin independent Wnt signaling involves multiple potentially overlapping signaling pathways, including Wnt-Calcium signaling and Planar Cell Polarity. These pathways have been especially difficult to parse in part because mWNT5A is the only non-canonical WNT available to the research community in the form of a WNT-producing cell line (Chen W et al, Science 301:1391-1394 (2003)). Applicants thus generated hWNT11 and hWNT16 expressing iCHO cell lines to enable further study of beta-catenin-independent Wnt signaling mechanisms. Like hWNT5A, neither hWNT11 nor hWNT16 induced STF in co-culture (FIG. 2E) and both inhibited hWNT3A activity to a similar degree (FIG. 2F).

### Example 3

### Purification of Bioactive Human Wnt Proteins

Applicants used methods for the purification of mWNT3A from L cell conditioned media (Willert K et al., Nature 423:448-452 (2003)) to purify mWNT3A from iCHO conditioned media and found iCHO cells to be a superior source of mWNT3A protein. Not only did iCHO cells produce and secrete more mWNT3A than L cells (FIG. 6B), but collection and filtration of iCHO conditioned media was more efficient because iCHO cells did not detach from the plate at high cell densities, permitting successive harvests of conditioned media from an expanded population. Furthermore, iCHO conditioned media did not clog the filters during removal of dead cells and debris, a common issue with L cell conditioned media (data not shown).

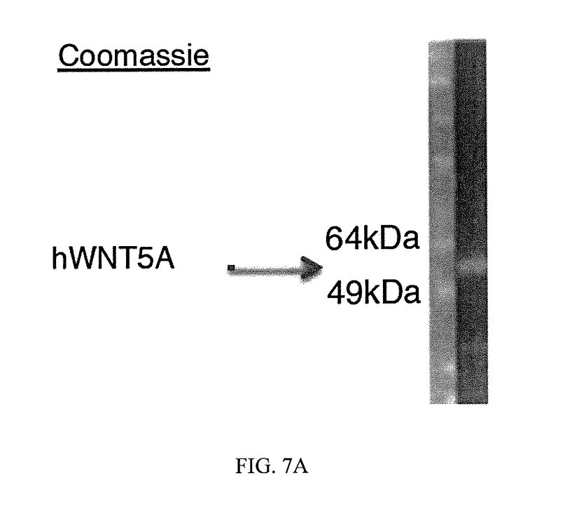

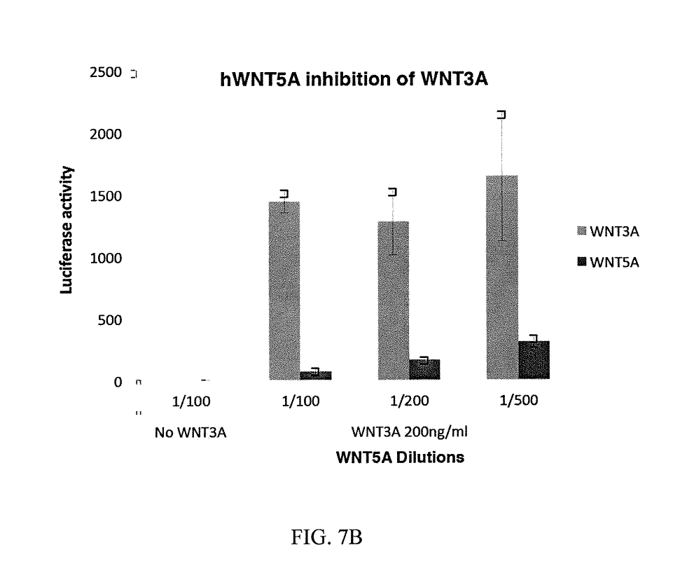

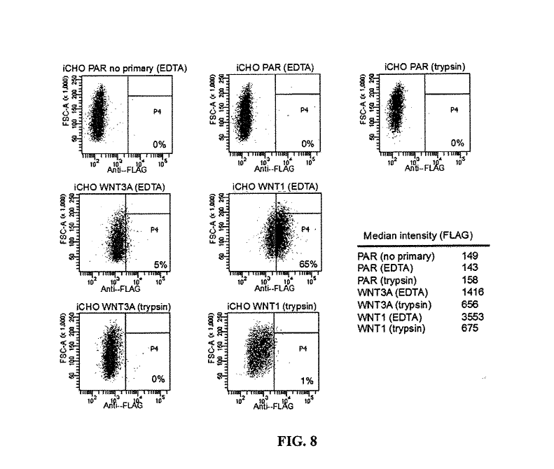

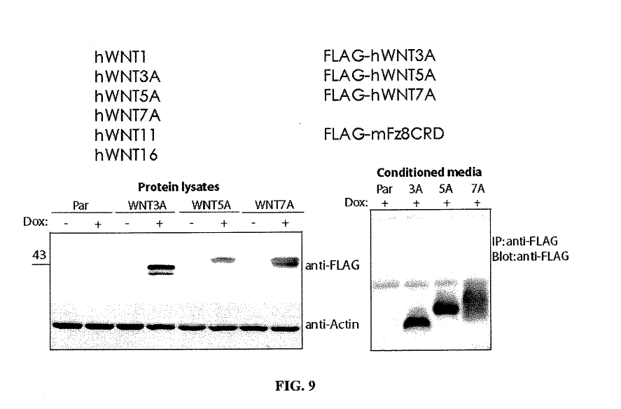

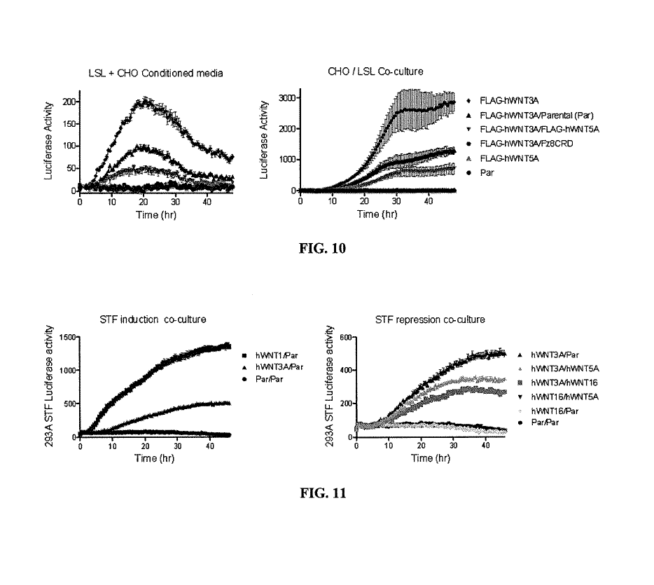

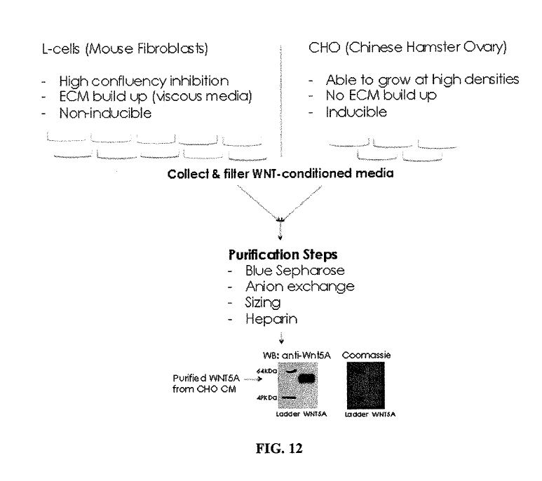

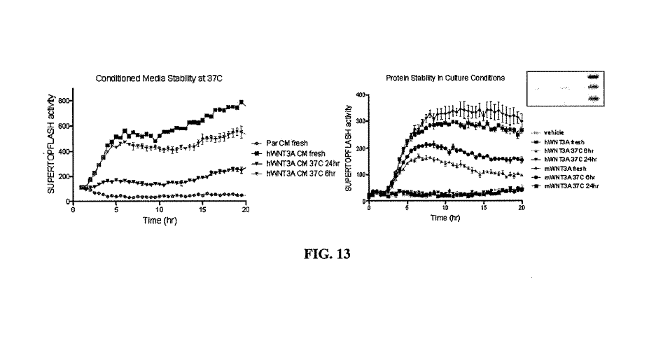

Applicants successfully used the same method to purify human WNTs (hWNT3A and hWNT5A) from iCHO conditioned media. Both purified hWNT3A (FIG. 6C, D) and hWNT5A (FIG. 7) displayed the expected activity. Purified hWNT3A from iCHO conditioned media and mWNT3A from L conditioned media migrated at the same position on an SDS-polyacrylamide gel (FIG. 6C). While this suggests that the two proteins share similar post-translational modifications, mWNT3A displayed slightly higher activity. Both mWNT3A and hWNT3A rapidly lost activity when pre-incubated in culture media at 37° C. (FIG. 6D), suggesting that continuous production in a co-culture environment with iCHO cells may be advantageous when sustained Wnt signaling is required.

### Example 4

### Distinct Properties of Different Canonical Wnt Proteins

Although WNT3A is widely considered as representative of all canonical Wnts, it was recently reported that canonical Wnts can be subdivided into three groups based on their interaction with the LRP6 co-receptor (Ettenberg S A et al., Proceedings of the National Academy of Sciences of the United States of America 107:15473-15478 (2010), Gong Y et al., PloS one 5: e12682 (2010)). Applicants thus generated additional iCHO cell lines expressing hWNT1 and hWNT7A to represent each of the other groups.

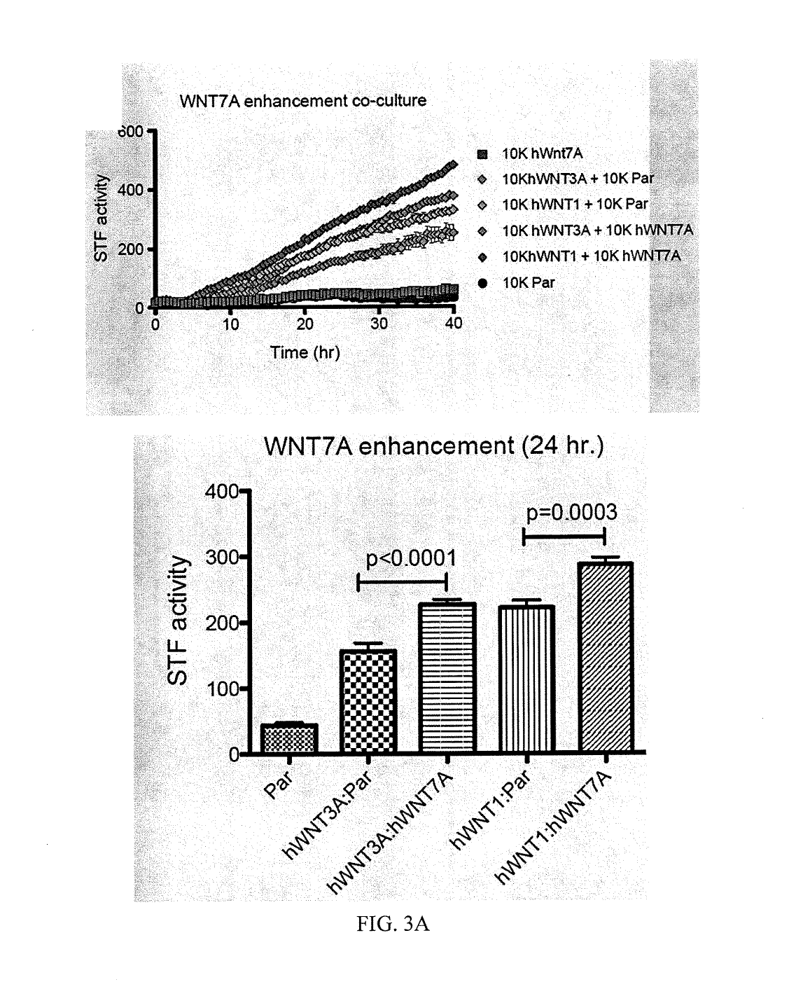

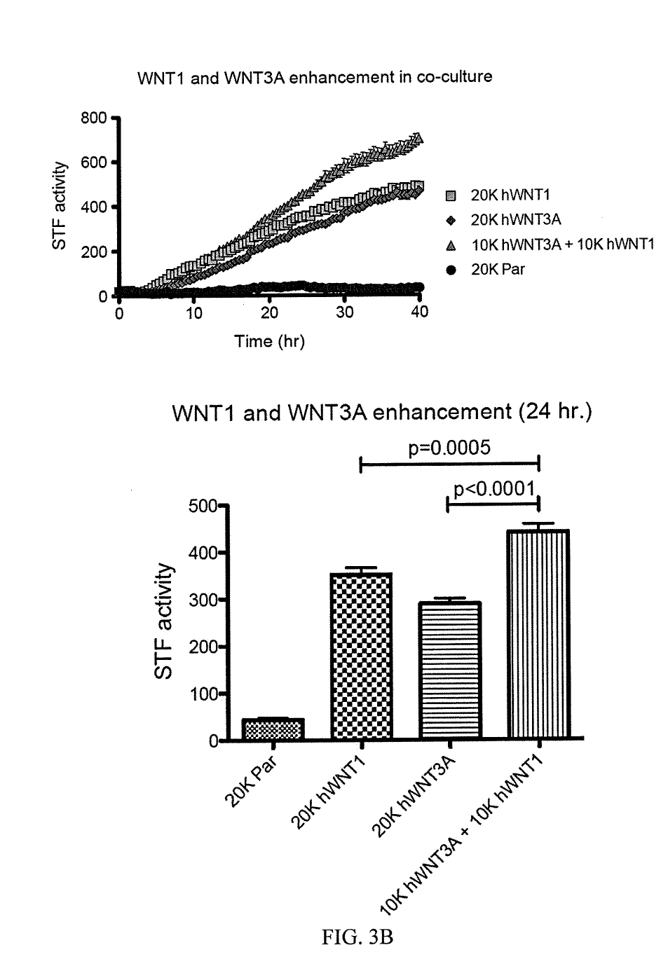

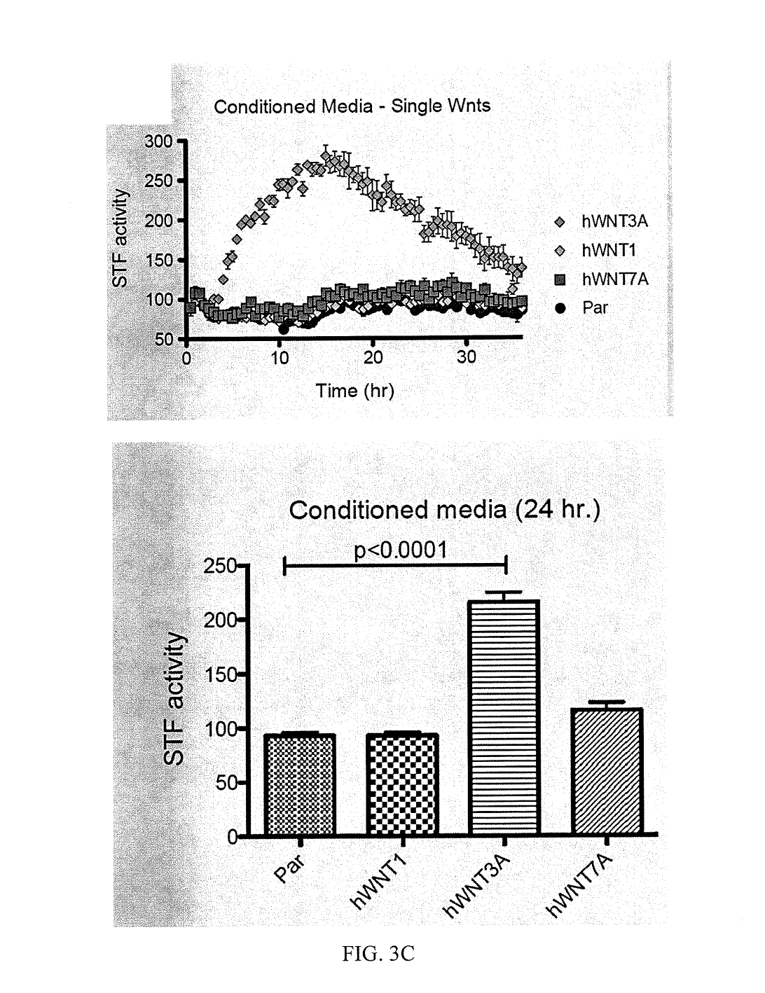

Among the canonical Wnts, Applicants found that both hWNT3A and hWNT1 robustly activate STF in co-culture assays (FIG. 2E). hWNT7A alone exhibited weak and delayed induction of STF, but showed clear and reproducible enhancement of hWNT3A and hWNT1 activity in combinatorial assays in which the total number of iCHO-Wnt cells in each condition was constant (FIG. 3A). Applicants also observed enhanced STF induction when hWNT1 and hWNT3A were introduced together, consistent with recent reports that these two Wnts employ parallel signaling mechanisms (FIG. 3B) (Ettenberg S A et al., Proceedings of the National Academy of Sciences of the United States of America 107:15473-15478 (2010); Gong Y et al., PloS one 5: e12682 (2010)).

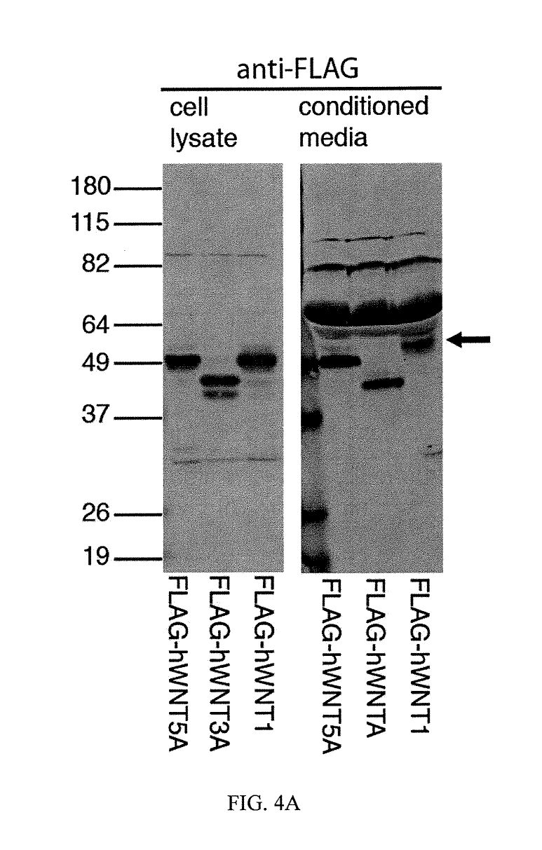

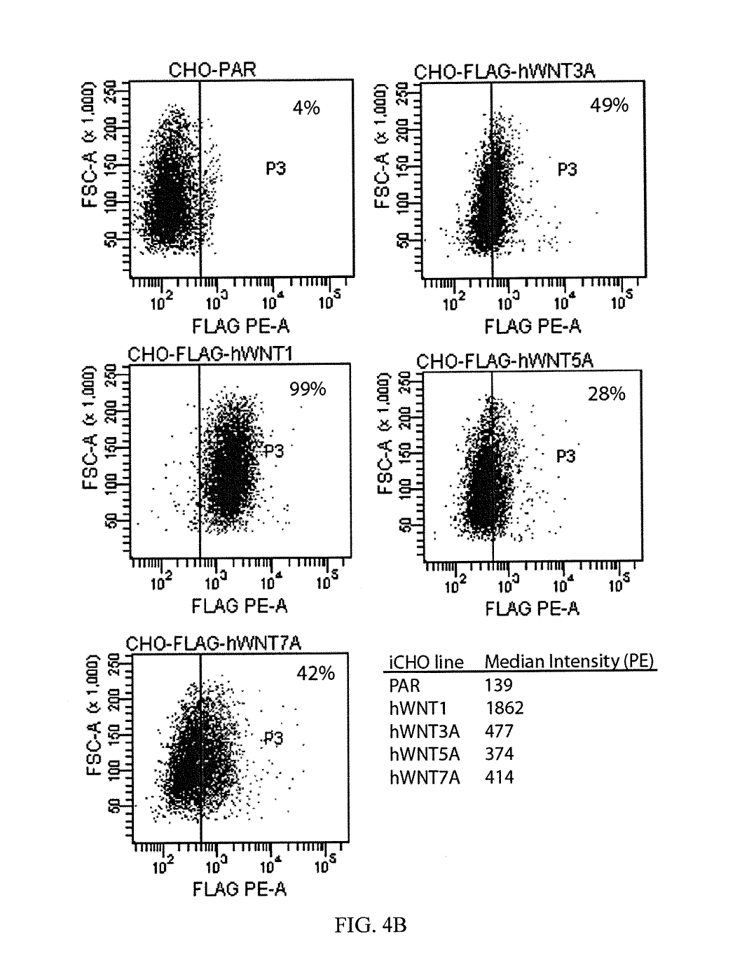

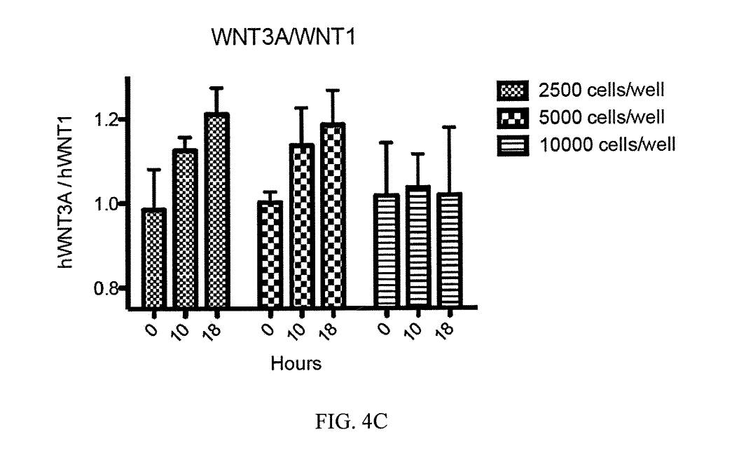

hWNT3A conditioned media robustly activated STF, although the magnitude was lower than that observed with co-culture (FIG. 3C). hWNT5A likewise had activity in conditioned media (FIG. 2A). In contrast, while hWNT1 was a potent inducer in co-culture, hWNT1 conditioned media failed to induce STF. This difference in activity could be due to reduced expression or secretion of hWNT1 compared to the other WNTs. Applicants thus generated iCHO cells expressing FLAG-hWNT1 and used the common FLAG epitope to compare protein levels of FLAG-hWNT1 to FLAG-hWNT3A and FLAG-hWNT5A in iCHO cell lysates and conditioned media. Although all three WNTs were present in cell lysates at similar levels, there was less FLAG-hWNT1 present in the conditioned media and the FLAG-hWNT1 protein that was detected in conditioned media migrated slower than the corresponding protein in the cell lysate (FIG. 4A). Lower protein levels and lack of activity of hWNT1 in the conditioned media suggested that WNT1 may act more locally than WNT3A, which retains activity in conditioned media. Localized function of WNT1 would require that signaling and responding cells be in close proximity, possibly requiring cell-to-cell contact, leading Applicants to hypothesize that WNT1 may be preferentially retained on the surface of the producing cell, compared to WNT3A. Flow cytometry using the FLAG epitope confirmed that there was more FLAG-hWNT1 on the iCHO cell surface than FLAG-hWNT3A, FLAG-hWNT5A or FLAG-hWNT7A, which were all present at similar levels (FIG. 4B). Applicants next performed a co-culture dilution experiment to compare the range of activity between hWNT1 and hWNT3A. Applicants reasoned that if WNT3A travels freely in the extracellular space and WNT1 does not, then at lower densities WNT3A should accumulate in the media over time, inducing reporter expression in responding cells at a greater rate than WNT1. At higher densities, when cell-to-cell contact is saturated, the rate of reporter induction should be similar between WNT1 and WNT3A. This is indeed what Applicants observed (FIG. 4C). Together, these results indicate that WNT1 activity requires that cells be in close proximity while WNT3A is better suited to act at a distance.

The present invention provides an improved and highly efficient RMCE method to engineer transgenic iCHO cells to produce proteins of biologic interest. Applicants provide proof-of-principle by applying this method to generate a family of cell lines that inducibly express human Wnt proteins or a Wnt antagonist. Applicants chose the Wnt family of proteins to exemplify the robustness and versatility of this strategy because Wnt proteins are notoriously challenging to work with and difficult to obtain.

Applicants developed a kinetic Wnt reporter assay to characterize the biological activity of the proteins generated by each cell line. Using this assay, Applicants found variability in the peak of Wnt-induced luciferase activity measured for different cell lines responding to the same Wnt. For example, maximal response to hWNT3A of a L cell STF reporter line occurred at around 20 hours (FIG. 2A), while a 293A-based reporter system occurred closer to 15 hours post treatment (FIG. 3C). Although most current studies measure Wnt signaling by endpoint STF luciferase assays, the method shows that critical effects can be missed if a time point is chosen in advance of, or following, the peak. Thus, the kinetic assay described here provides a more comprehensive and reliable means of evaluating Wnt activity.

Applicants noted that while co-culture with Wnt-producing cells induced a sustained STF response, conditioned media produced a response that peaked and then declined within the assay period. This could be due to the Wnt in the conditioned media being depleted, to receptor turnover in the responding cells, or to a loss of Wnt protein activity over time. By pre-incubating the protein at 37° C. prior to treating the cells, Applicants demonstrated that WNT3A loses much of its activity after 6 hours and is completely inactive after 24 hours. Thus, long-term experiments with Wnt proteins would require regular reintroduction of fresh protein to maintain signaling. This presents a technical challenge because purified Wnt protein is stored in a high detergent buffer and accumulation of this buffer can be toxic to cells. Where possible, co-culture can be an easy and affordable alternative to maintain a steady supply of Wnt in the media.

Among the non-canonical Wnts, Applicants found that hWNT5A, hWNT11 and hWNT16 each inhibited hWNT3A activity to a similar degree. The ability of WNT5A and WNT11 to inhibit canonical Wnt signaling is well established (Mikels A J, Nusse R, PLoS biology 4:e115 (2006); Uysal-Onganer P, Kypta R M, Acta Physiol (Oxf) (2011); Veeman M T et al., Developmental cell 5:367-377 (2003a)) however, to Applicants' knowledge, a similar function of WNT16 has not been previously described. Given recent evidence that WNT16 plays a role in the specification of hematopoietic stem cells and in human leukemia, it is important to elucidate such mechanistic properties of Wnt16 signaling (Clements W K et al., Nature 474:220-224 (2011); Mazieres J et al., Oncogene 24:5396-5400 (2005); McWhirter J R et al., Proceedings of the National Academy of Sciences of the United States of America 96:11464-11469 (1999); Lu et al., PNAS 2003).

While both hWNT1 and hWNT3A displayed similar activity in co-culture, only hWNT3A exhibited activity using conditioned media. This is consistent with previous reports using fibroblasts expressing mWNT1 where it was shown that the majority of this protein was associated with the extracellular matrix, and that little or none was detectable in the conditioned media (Bradley R S, Brown A M, The EMBO journal 9:1569-1575 (1990)). This phenomenon has been reported for other WNT1-expressing cell types as well (Papkoff J et al., Molecular and cellular biology 7:3978-3984 (1987); Papkoff J., Molecular and cellular biology 9:3377-3384 (1989)), but was never compared to other WNTs in the same system to show that poor secretion is specific to WNT1. Using flow cytometry and FLAG-tagged Wnt proteins Applicants further showed that WNT1 is retained on the cell surface to a much greater extent than WNT3A or the other Wnts tested, suggesting that WNT1 has a more limited range of activity than WNT3A. These differences in secretion and membrane retention could reflect different biological functions whereby WNT1 is effective at proximal intercellular communication, possibly requiring cell:cell contact, while WNT3A behaves more like a classical morphogen able to act over a distance.

Canonical Wnt signaling is triggered when a Wnt protein binds to Frizzled and the LRP5 or LRP6 co-receptor. Recent studies showed that canonical Wnts could be divided into three classes based upon their interaction with LRP6 (Ettenberg S A et al., Proceedings of the National Academy of Sciences of the United States of America 107:15473-15478 (2010); Gong Y et al., PloS one 5: e12682 (2010)). WNT1 class proteins (WNT1, 2, 2b, 6, 8a, 9a, 9b, 10b) bind to the first YWTD-type beta-propeller domain of LRP6 and WNT3A class proteins (WNT3, 3a) bind to the third propeller of LRP6. Anti-LRP6 antibodies specific to these propeller domains can specifically block WNT1 class or WNT3A class activity. A third class of Wnt proteins (WNT4, 7a, 7b, 10a) is not inhibited by antibodies against either domain, suggesting that they function by a different mechanism. Interestingly, Wnts from the WNT1 and WNT3A classes can bind to LRP6 simultaneously (Bourhis et al., 2010). This parallel signaling mechanism could explain why Applicants observe greater activity when hWNT1 and hWNT3A are introduced together, than when either is introduced alone. Furthermore, Applicants' observation that hWNT7A enhanced both hWNT1 and hWNT3A activity is consistent with yet another parallel signaling mechanism for this class of Wnt proteins.

Using the compositions and methods provided herein including embodiments thereof combinatorial and comparative studies of Wnt proteins that are now possible. Placement of the Wnt-expression transgenes in identical genomic loci enables such head-to-head comparisons that are currently impossible because of inconsistencies in quality between protein preparations, variable expression between Wnt-producing cell lines or a complete unavailability of many Wnts.

The eleven Wnt-producing iCHO cell lines described here comprise a powerful resource for further analysis of Wnt activity in diverse settings. These lines will help mitigate the current lack of adequate sources of certain Wnt proteins, including those of human origin, and should expedite progress in critical research areas where Wnt function is either heavily implicated or poorly understood, such as regenerative medicine, stem cell biology, and cancer research (Clevers H, Cell 127:469-480 (2006)). Moreover, the facile and robust method Applicants developed to generate these lines will expedite generation of additional cell lines expressing other Wnts and their inhibitors to enable a deeper understanding of the mechanisms underlying this complex set of regulatory proteins. Furthermore, the low background expression and high inducibility of this system make it a molecular genetic platform technology attractive for the production of diverse classes of proteins, including those that are toxic when overexpressed, and those requiring association with the producing cell for their biologic activity. Therefore, this system has the potential to accelerate analysis of any signaling protein that is currently limited by protein production.

Experimental Procedures

Molecular Biology. The following Wnt cDNAs were used in the Wnt expression vectors, most were obtained from OpenBiosystems: hWNT1 (accession number BC074799), hWNT3A (BC103921), hWNT5A (BC064694), hWNT7A (BC008811), hWNT11 (BC074791), hWNT16 (BC104945). All FLAG-tagged WNT proteins contain the hWNT3A signal sequence followed by a FLAG tag. A BspEI restriction site was used to join the FLAG tag with the remaining Wnt cDNA. Untagged Wnts were cloned into plasmid pJG011 (L3-pTRETight-eGFP-polyA-SV40-BSD-2L) using BamHI 5′ and either NotI or NheI 3′. pTRE-Tight was from Clontech.

Cell Culture. Parental CHO cells were propagated in DMEM, 10% FBS, 0.4 mg/mL G418 (to maintain the rtTA transgene). Post-RMCE CHO cells were propagated in the above media plus 3 μg/mL Blasticidin and 5 ng/mL Doxycycline. 293A cells were co-transfected with the STF plasmid (gift from RT Moon) and the pcDNA3.1 His/lacZ plasmid (Life Technologies) in a 1:6 ratio and selected with 1.2 mg/mL G418. LSL cells (gift from Roel Nusse) were propagated in DMEM, 10% FBS.

RMCE. Parental CHO cells were transfected with PEI in a 6 well plate with 2 μg total of the incoming exchange plasmid and the Cre recombinase plasmid (pOG231) at a 2:1 ratio. Media was changed after 6 hr. The following day, cells were trypsinized and expanded to 15 cm plates at low density. The following day, cells were treated with 2 μM Ganciclovir. Three days later media was replaced with fresh Ganciclovir-containing media. Four to seven days later, Gan-resistant colonies emerged and all cells on negative control plate were dead. At this point, Ganciclovir-containing media was replaced with media containing 3 μg/mL Blasticidin plus 5 ng/mL Dox (in Applicants' experience more colonies emerge with concomitant addition of Dox, possibly due to opening of the genomic locus where exchange occurs). True colonies emerged after four days of Blasticidin selection. Colonies were then picked and expanded.

Western blotting and immunoprecipitation (IP). Equal amounts of total protein from cell lysates and equal volumes of unconcentrated conditioned media were separated by SDS PAGE using standard methods. Primary antibodies used were mouse anti-FLAG M2 (Sigma) and rabbit anti-FLAG (gift from Peter Gray, Salk). Secondary antibodies were conjugated to AF-680 (Life Technologies) or IRDye800 (Rockland) for scanning with LiCOR Odyssey. To prepare conditioned media (CM) for IP, 3.5M CHO cells were seeded in a 10 cm dish in drug-free media plus 250 ng/mL Dox. 72 hours later, media was collected, filtered (0.2 μm) and stored at 4° C. for less than one week before use. For IP, anti-FLAG sepharose (Sigma) was incubated overnight with CM, washed, resuspended in sample buffer with DTT, boiled, and used for SDS PAGE.

Real-time bioluminescence monitoring assays. Twenty thousand 293A-STF cells were seeded into a 96 well white opaque plate (Corning) with or without twenty thousand engineered CHO cells in phenol red-free DMEM-F12 (Life Technologies), 10% serum, 100 μM D-Luciferin (Biosynth) and 250 ng/mL Dox. Real-time luminescence counts from each well were collected every 30 minutes by a temperature-controlled luminometer (Tecan M200) set to 37° C. While the background signal from parental CHO cells was relatively consistent from assay to assay, the level of induction was variable, depending on the reporter cells and plate format used. For this reason, and because the background readings are extremely low, Applicants were unable to calculate a normalized induction for cross-comparison between experiments.

Wnt purification. Wnt proteins were purified from 2-6 liters of iCHO conditioned media (CM). CM was complemented with 1% Triton X-100, 20 mM Tris-HCl pH7.5 and 0.01% NaN₃. The purification method consists of four consecutive steps performed on an Äkta purifier (GE Healthcare) in the presence of 1% CHAPS. In the first step, Wnts were bound on Blue Sepharose (GE Healthcare) and fractions were eluted with increasing KCl concentrations. Wnt containing fractions were then bound to a copper-chelated resin (GE Healthcare) and eluted with increasing concentrations of Imidazole. Partially purified Wnts were then separated by gel filtration on Superdex 200 pg (GE Healthcare). Wnt fractions were further purified by Heparin affinity chromatography (GE Healthcare). Wnt yields assessed by Coomassie staining average 40 μg/L.

Flow cytometry. iCHO cells were grown for 48 hours with 250 ng/mL Dox, harvested with EDTA and stained with anti-FLAG primary antibody (M2, Sigma) and anti-mouse secondary antibody AF568 (Life Technologies).

### REFERENCES

Ashe H L, Briscoe J, Development 133:385-394 (2006).
Bradley R S, Brown A M, The EMBO journal 9:1569-1575 (1990).
Cajanek L et al., Journal of cellular biochemistry 111:1077-1079 (2010).
Carmon K S et al., Proceedings of the National Academy of Sciences of the United States of America 108:11452-11457 (2011).
Chen W et al, Science 301:1391-1394 (2003).
Clements W K et al., Nature 474:220-224 (2011).
Clevers H, Cell 127:469-480 (2006).
Coudreuse D, Korswagen H C, Development 134:3-12 (2007).
de Lau W et al., Nature 476:293-297 (2011).
Ettenberg S A et al., Proceedings of the National Academy of Sciences of the United States of America 107:15473-15478 (2010).
Glinka A et al., EMBO reports 12:1055-1061 (2011).
Gong Y et al., PloS one 5: e12682 (2010).
Hsieh J C et al., Proceedings of the National Academy of Sciences of the United States of America 96:3546-3551 (1999).
Jue S F et al., Molecular and cellular biology 12:321-328 (1992).
Korinek V et al., Science 275:1784-1787 (1997).
Mazieres J et al., Oncogene 24:5396-5400 (2005).
McWhirter J R et al., Proceedings of the National Academy of Sciences of the United States of America 96:11464-11469 (1999).
Mikels A J, Nusse R, PLoS biology 4:e115 (2006).
Papkoff J et al., Molecular and cellular biology 7:3978-3984 (1987).
Papkoff J., Molecular and cellular biology 9:3377-3384 (1989).
Pulivarthy S R et al., Proceedings of the National Academy of Sciences of the United States of America 104:20356-20361 (2007).
Reya T et al., Nature 423:409-414 (2003).
Uysal-Onganer P, Kypta R M, Acta Physiol (Oxf) (2011).
van Amerongen R, Nusse R, Development 136:3205-3214 (2009).
Veeman M T et al., Developmental cell 5:367-377 (2003a).
Veeman M T et al., Current biology: CB 13:680-685 (2003b).
Walsh G, Nature biotechnology 28:917-924 (2010).
Willert K et al., Nature 423:448-452 (2003).
Wong E T et al., Nucleic acids research 33:e147 (2005).
Wurm F M, Nature biotechnology 22:1393-1398 (2004).

Unless indicated otherwise, accession numbers provided herein refer to biomolecular entries from the National Center for Biotechnology Information (NCBI) (U.S. National Library of Medicine, Bethesda, Md.), each of which is incorporated by reference herein in its entirety and for all purposes.

What is claimed is:

1. A method of expressing a protein in a cell, the method comprising:
(i) transfecting a stably transfected recombination cell with an expression nucleic acid and a recombinase nucleic acid, thereby forming an expression cell, wherein said expression nucleic acid comprises a protein encoding nucleic acid sequence and a recombination selection nucleic acid; and
(ii) allowing said expression cell to express said protein encoding nucleic acid, thereby expressing said protein.

2. The method of claim 1, wherein said stably transfected recombination cell is a mammalian cell.

3. The method of claim 2, wherein said mammalian cell is an adherent cell.

4. The method of claim 1, wherein said expression nucleic acid further comprises a promoter nucleic acid sequence operably linked to said protein encoding nucleic acid sequence.

5. The method of claim 4, wherein said promoter nucleic acid sequence is an inducible promoter nucleic acid sequence.

6. The method of claim 1, wherein said expression nucleic acid forms part of a first nucleic acid and said recombinase nucleic acid forms part of a second nucleic acid.

7. The method of claim 1, wherein said method further comprises after said transfecting of step (i) and before said allowing of step (ii), a step (i.a) of administering to said expression cell a negative selection substrate.

8. The method of claim 7, wherein said method further comprises after said administering of (i.a), a step (i.b) of administering a recombination selection substrate.

9. The method of claim 8, wherein said administering of (i.b) further comprises administering a promoter inducing compound.

10. The method of claim 1, wherein said protein is a Wnt protein and said protein encoding nucleic acid sequence is a Wnt protein encoding nucleic acid sequence.

11. The method of claim 10, wherein said Wnt protein encoding nucleic acid sequence comprises SEQ ID:1, SEQ ID:2, SEQ ID:3, SEQ ID:4, SEQ ID:5, or SEQ ID:6.

12. The method of claim 10, further comprising:
(iii) separating said Wnt protein from said expression cell, thereby preparing a free Wnt protein.

13. The method of claim 12, wherein said free Wnt protein is a secreted Wnt protein.

14. The method of claim 13, wherein said free Wnt protein is an intracellular Wnt protein.

15. A stably transfected recombination cell comprising an expression nucleic acid and a recombinase nucleic acid, wherein said expression nucleic acid comprises a protein encoding nucleic acid sequence and a recombination selection nucleic acid.

16. The stably transfected recombination cell of claim 15, wherein said expression nucleic acid further comprises a promoter nucleic acid sequence operably linked to said protein encoding nucleic acid sequence.

17. The stably transfected recombination cell of claim 15, wherein said protein encoding nucleic acid sequence is a Wnt protein encoding nucleic acid sequence.

18. The stably transfected recombination cell of claim 17, wherein said Wnt protein encoding nucleic acid sequence is the sequence of SEQ ID:1, SEQ ID:2, SEQ ID:3, SEQ ID:4, SEQ ID:5, or SEQ ID:6.

19. An expression cell comprising a genome integrated expression nucleic acid, said genome integrated expression nucleic acid comprising a protein encoding nucleic acid sequence and a recombination selection nucleic acid.

20. The expression cell of claim 19, wherein said expression cell is derived from a stably transfected recombination cell.

21. The expression cell of claim 19, wherein said expression nucleic acid further comprises a promoter nucleic acid sequence operably linked to said protein encoding nucleic acid sequence.

22. The expression cell of claim 19, wherein said protein encoding nucleic acid sequence is a Wnt protein encoding nucleic acid sequence.

23. The expression cell of claim 22, wherein said Wnt protein encoding nucleic acid sequence is the sequence of SEQ ID:1, SEQ ID:2, SEQ ID:3, SEQ ID:4, SEQ ID:5, or SEQ ID:6.


SEQUENCE LISTING (6 sequences)

SEQ ID NO: 1 - DNA, 1186 bp, Homo sapiens
caactgcagcacagagcgggcaaagccaggcaggccatggggctctgggcgctgttgcctggctgggtttctgctacgctgctgctggcgctggccgctctgcccgcagccctggctgccaacagcagtggccgatggtggggtattgtgaacgtagcctcctccacgaacctgcttacagactccaagagtctgcaactggtactcgagcccagtctgcagctgttgagccgcaaacagcggcgtctgatacgccaaaatccggggatcctgcacagcgtgagtggggggctgcagagtgccgtgcgcgagtgcaagtggcagttccggaatcgccgctggaactgtcccactgctccagggccccacctcttcggcaagatcgtcaaccgaggctgtcgagaaacggcgtttatcttcgctatcacctccgccggggtcacccattcggtggcgcgctcctgctcagaaggttccatcgaatcctgcacgtgtgactaccggcggcgcggccccgggggccccgactggcactgggggggctgcagcgacaacattgacttcggccgcctcttcggccgggagttcgtggactccggggagaaggggcgggacctgcgcttcctcatgaaccttcacaacaacgaggcaggccgtacgaccgtattctccgagatgcgccaggagtgcaagtgccacgggatgtccggctcatgcacggtgcgcacgtgctggatgcggctgcccacgctgcgcgccgtgggcgatgtgctgcgcgaccgcttcgacggcgcctcgcgcgtcctgtacggcaaccgcggcagcaaccgcgcttcgcgggcggagctgctgcgcctggagccggaagacccggcccacaaaccgccctccccccacgacctcgtctacttcgagaaatcgcccaacttctgcacgtacagcggacgcctgggcacagcaggcacggcagggcgcgcctgtaacagctcgtcgcccgcgctggacggctgcgagctgctctgctgcggcaggggccaccgcacgcgcacgcagcgcgtcaccgagcgctgcaactgcaccttccactggtgctgccacgtcagctgccgcaactgcacgcacacgcgcgtactgcacgagtgtctgtgaggcgctgcgcggactcgcccccaggaaacgctctcct

SEQ ID NO: 2 - DNA, 1369 bp, Homo sapiens
acgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaaggt

SEQ ID NO: 3 - DNA, 5855 bp, Homo sapiens
agttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagattttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattatcccaaaagttttttgagtcttgagcttgggctgtggccctgctgtgatcataccttgagcacaacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttgctgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctgcacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgattaagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatgtcttgtacttttctattaaaatcattttatgaaatccaaaaaaaaaaaaaaa

SEQ ID NO: 4 - DNA, 1732 bp, Homo sapiens
gaggggcgggggctggaggcagcagcgcccccgcactccccgcgtctcgcacacttgcaccggtcgctcgcgcgcagcccggcgtcgccccacgccgcgctcgctcctccctccctcctcccgctccgtggctcccgtgctcctggcgaggctcaggcgcggagcgcgcggacgggcgcaccgacagacggccccggggacgcctcggctcgcgcctcccgggcgggctatgttgattgccccgccggggccggcccgcgggatcagcacagcccggcccgcggccccggcggccaatcgggactatgaaccggaaagcgcggcgctgcctgggccacctctttctcagcctgggcatggtctacctccggatcggtggcttctcctcagtggtagctctgggcgcaagcatcatctgtaacaagatcccaggcctggctcccagacagcgggcgatctgccagagccggcccgacgccatcatcgtcataggagaaggctcacaaatgggcctggacgagtgtcagtttcagttccgcaatggccgctggaactgctctgcactgggagagcgcaccgtcttcgggaaggagctcaaagtggggagccgggaggctgcgttcacctacgccatcattgccgccggcgtggcccacgccatcacagctgcctgtacccagggcaacctgagcgactgtggctgcgacaaagagaagcaaggccagtaccaccgggacgagggctggaagtggggtggctgctctgccgacatccgctacggcatcggcttcgccaaggtctttgtggatgcccgggagatcaagcagaatgcccggactctcatgaacttgcacaacaacgaggcaggccgaaagatcctggaggagaacatgaagctggaatgtaagtgccacggcgtgtcaggctcgtgcaccaccaagacgtgctggaccacactgccacagtttcgggagctgggctacgtgctcaaggacaagtacaacgaggccgttcacgtggagcctgtgcgtgccagccgcaacaagcggcccaccttcctgaagatcaagaagccactgtcgtaccgcaagcccatggacacggacctggtgtacatcgagaagtcgcccaactactgcgaggaggacccggtgaccggcagtgtgggcacccagggccgcgcctgcaacaagacggctccccaggccagcggctgtgacctcatgtgctgtgggcgtggctacaacacccaccagtacgcccgcgtgtggcagtgcaactgtaagttccactggtgctgctatgtcaagtgcaacacgtgcagcgagcgcacggagatgtacacgtgcaagtgagccccgtgtgcacaccaccctcccgctgcaagtcagattgctgggaggactggaccgtttccaagctgcgggctccctggcaggatgctgagcttgtcttttctgctgaggagggtacttttcctgggtttcctgcaggcatccgtgggggaaaaaaaatctctcagagccctcaactattctgttccacacccaatgctgctccaccctcccccagacacagcccaggtccctccgcggctggagcgaagccttctgcagcaggaactctggacccctgggcctcatcacagcaatatttaacaatttattctgataaaaataatattaatttatttaattaaaaagaattcttccacaaaaaaaaaaaaaaaaaa

SEQ ID NO: 5 - DNA, 1154 bp, Homo sapiens
gctcctcctgggtgtgaccccgggcgcgcccgccgcgcgacgatgagggcgcggccgcaggtctgcgaggcgctgctcttcgccctggcgctccagaccggcgtgtgctatggcatcaagtggctggcgctgtccaagacaccatcggccctggcactgaaccagacgcaacactgcaagcagctggagggtctggtgtctgcacaggtgcagctgtgccgcagcaacctggagctcatgcacacggtggtgcacgccgcccgcgaggtcatgaaggcctgtcgccgggcctttgccgacatgcgctggaactgctcctccattgagctcgcccccaactatttgcttgacctggagagagggacccgggagtcggccttcgtgtatgcgctgtcggccgccgccatcagccacgccatcgcccgggcctgcacctccggcgacctgcccggctgctcctgcggccccgtcccaggtgagccacccgggcccgggaaccgctggggaggatgtgcggacaacctcagctacgggctcctcatgggggccaagttttccgatgctcctatgaaggtgaaaaaaacaggatcccaagccaataaactgatgcgtctacacaacagtgaagtggggagacaggctctgcgcgcctctctggaaatgaagtgtaagtgccatggggtgtctggctcctgctccatccgcacctgctggaaggggctgcaggagctgcaggatgtggctgctgacctcaagacccgatacctgtcggccaccaaggtagtgcaccgacccatgggcacccgcaagcacctggtgcccaaggacctggatatccggcctgtgaaggactcggaactcgtctatctgcagagctcacctgacttctgcatgaagaatgagaaggtgggctcccacgggacacaagacaggcagtgcaacaagacatccaacggaagcgacagctgcgaccttatgtgctgcgggcgtggctacaacccctacacagaccgcgtggtcgagcggtgccactgtaagtaccactggtgctgctacgtcacctgccgcaggtgtgagcgtaccgtggagcgctatgtctgcaagtgaggccctgccctccgccccacgcaggagcgaggactctgctcaaggac

SEQ ID NO: 6 - DNA, 1222 bp, Homo sapiens
tgcaaaagaggagcggctgggctgggggactccatgcgggggcgatggacagggcggcgctcctgggactggcccgcttgtgcgcgctgtgggcagccctgctcgtgctgttcccctacggagcccaaggaaactggatgtggttgggcattgcctccttcggggttccagagaagctgggctgcgccaatttgccgctgaacagccgccagaaggagctgtgcaagaggaaaccgtacctgctgccgagcatccgagagggcgcccggctgggcattcaggagtgcgggagccagttcagacacgagagatggaactgcatgatcaccgccgccgccactaccgccccgatgggcgccagccccctctttggctacgagctgagcagcggcaccaaagagacagcatttatttatgctgtgatggctgcaggcctggtgcattctgtgaccaggtcatgcagtgcaggcaacatgacagagtgttcctgtgacaccaccttgcagaacggcggctcagcaagtgaaggctggcactgggggggctgctccgatgatgtccagtatggcatgtggttcagcagaaagttcctagatttccccatcggaaacaccacgggcaaagaaaacaaagtactattagcaatgaacctacataacaatgaagctggaaggcaggctgtcgccaagttgatgtcagtagactgccgctgccacggagtttccggctcctgtgctgtgaaaacatgctggaaaaccatgtcttcttttgaaaagattggccatttgttgaaggataaatatgaaaacagtatccagatatcagacaaaacaaagaggaaaatgcgcaggagagaaaaagatcagaggaaaataccaatccataaggatgatctgctctatgttaataagtctcccaactactgtgtagaagataagaaactgggaatcccagggacacaaggcagagaatgcaaccgtacatcagagggtgcagatggctgcaacctcctctgctgtggccgaggttacaacacccatgtggtcaggcacgtggagaggtgtgagtgtaagttcatctggtgctgctatgtccgttgcaggaggtgtgaaagcatgactgatgtccacacttgcaagtaaccactccatccagccttgggcaagatgcctcagcaatatacaatggcattgcaaccagagaggtgcccatccctgtgcag